SEQUENCE LISTING (3 sequences)

SEQ ID NO: 1 - DNA, 25 bp, artificial sequence
  Primer sequence
agtctggcttatatccaacacttcg

SEQ ID NO: 2 - DNA, 22 bp, artificial sequence
  primer sequence
gactttgctttccttggtcagg

SEQ ID NO: 3 - DNA, 26 bp, artificial sequence
  primer sequence
tttcaccagcaagcttgcgaccttga

(19) United States
(12) Patent Application Publication
(10) Pub. No.: US 2013/0143223 A1
(43) Pub. Date: Jun. 6, 2013

(54) METHOD OF DETERMINING KIDNEY TRANSPLANTATION TOLERANCE

(71) Applicants: Maria Hernandez-Fuentes, London (GB); Irene Rebollo-Mesa, London (GB); Uwe Janssen, Gladbach (DE); Stefan Tomiuk, Gladbach (DE); Birgit Sawitzki, Berlin (DE); Hans-Dieter Volk, Berlin (DE); Robert Lechler, London (GB)

(72) Inventors: Maria Hernandez-Fuentes, London (GB); Irene Rebollo-Mesa, London (GB); Uwe Janssen, Gladbach (DE); Stefan Tomiuk, Gladbach (DE); Birgit Sawitzki, Berlin (DE); Hans-Dieter Volk, Berlin (DE); Robert Lechler, London (GB)

(21) Appl. No.: 13/696,215

(22) PCT Filed: May 4, 2011
(86) PCT No.: PCT/GB2011/050874

§ 371 Date: Jan. 29, 2013

(30) Foreign Application Priority Data
May 4, 2010 (GB) 1007454.0

(51) Int. Cl. C12Q 1/68

(52) U.S. Cl. CPC C12Q 1/6876
USPC 435/6.12

(57) ABSTRACT

The present invention relates to a method of determining an individual's transplantation tolerance by determining the level of a number of biomarkers. The present invention also relates to a kit comprising reagents for detecting the levels of the biomarkers. The present invention also relates to a sensor for detecting the expression levels of a plurality of genes that can be used to determine an individual's transplantation tolerance.

The present invention relates to a method of determining an individual's transplantation tolerance by determining the level of a number of biomarkers. The present invention also relates to a kit comprising reagents for detecting the levels of the biomarkers. The present invention also relays to a sensor for detecting the expression levels of a plurality of genes that can be used to determine an individual's transplantation tolerance.

Transplantation tolerance is defined as the stable maintenance of good allograft function in the sustained absence of immunosuppressive therapy. In the clinical arena it is only visible when patients experience stable allograft function despite having ceased all immunosuppression for an extended period of time. This state, defined as operational tolerance, has rarely been reported in renal transplantation (1-5), being more common in liver transplantation (6, 7). Long term survival of kidney transplants currently depends on sustained drug-induced immunosuppression. However, this is accompanied by increased morbidity and mortality, mainly due to cardiovascular disease, opportunistic infection and malignancy (8). Currently, we do not have the means to identify a priori those patients who are developing tolerance to their transplants and who would therefore benefit from partial or complete cessation of immunosuppression. Hence, there is an increasing need to develop assays and identify biomarkers that would allow clinicians to safely minimise immunosuppression, based on a patient's specific immunological profile.

Previous studies have identified biomarkers of tolerance in liver transplant recipients. In particular, cytokine gene polymorphisms were studied in a cohort of paediatric recipients. All of the immunosuppression-free children and the majority of those on minimal immunosuppression displayed low tumour necrosis factor (TNF)-α and high/intermediate interleukin (IL)-10 profiles in comparison with control patients on maintenance immunosuppression (36). In addition there was a difference in dendritic cell subset ratios between the two groups of patients. In comparison with patients on maintenance immunosuppression, circulating levels of plasmacytoid dendritic cells (pDC2), reported to selectively induce T-helper (Th) 2 responses, were more prevalent relative to monocytoid dendritic cells (pDCD1), which induce Th1-type responses, in the immunosuppression-free or minimally immunosuppressed patients (37).

A further attempt at identifying biomarkers of tolerance in adult liver transplant recipients using peripheral blood gene expression profiling and extensive blood cell immunophenotyping has been performed. It was demonstrated that operationally tolerant patients could be identified with a signature of genes that encoded γδT cell and natural killer (NK) cell receptors, as well as genes involved in cell proliferation arrest (38). They also found in the tolerant patients greater numbers of circulating potentially regulatory T-cell subsets, CD4⁺CD25⁺ T-cells and γδ T cells, in particular the Vδ1⁺ sub-type that has been implicated in immunoregulatory processes in epithelial tissues. Interestingly, previously observed differences in ratios of dendritic cell subsets could not be replicated in this patient cohort. The same group have studied gene expression profiles in the peripheral blood of liver transplant recipients comparing patients where immunosuppression weaning was successful with those where the weaning process was attempted but led to acute rejection requiring reintroduction of immunosuppression and with healthy controls (39). They identified three distinct gene signatures incorporating a modest number of genes (between 2 and 7) that discriminated tolerant and non-tolerant liver allograft recipients and healthy non-transplanted controls. This genomic footprint of operational tolerance has been validated in an independent cohort of 23 additional liver transplant recipients and is mainly characterized by upregulation of genes encoding for a variety of cell-surface receptors expressed by NK, CD8⁺, and γδ T cells. The previously observed expansion of putative regulatory T cells (CD4⁺CD25⁺Foxp3⁺, γδTCR⁺, and δ1TCR⁺ T cells) in peripheral blood was replicated in this new set of tolerant recipients. Taken together it appears that a combination of transcriptional profiling and flow cytometry in peripheral blood may identify liver transplant recipients who are able to accept their grafts in the absence of pharmacological immunosuppression.

Soulillou et al analysed the TCR repertoire in five operationally tolerant kidney transplant recipients and demonstrated in these patients skewed TCR Vβ chain usage, observed mainly in the CD8⁺ subset. These cells were also characterized by a decrease in cytokine transcripts (IL10, IL2, IL13, IFN-γ), suggesting a state of hyporesponsiveness (40). There were in addition significantly fewer circulating CD8⁺CD28⁻ effector lymphocytes in tolerant patients in comparison with patients with chronic allograft rejection suggesting suppression of cytotoxicity in these patients (Baeten et al., 2005 (45)). Later the same group used expression arrays to identify a set of 33 genes that could correctly distinguish with high specificity operationally tolerant kidney transplant recipients from patients with acute and chronic allograft rejection and healthy age-matched volunteers (41). Expression of co-stimulatory genes and markers of early and late T cell activation were reduced in tolerant patients compared with controls, and although expression of the anti-inflammatory cytokine transforming growth factor-β (TGFβ) was not upregulated in tolerant patients, many TGFβ-regulated genes were.

The same group have analysed blood cell phenotypes and transcriptional patterns in a group of eight operationally tolerant kidney allograft recipients and demonstrated higher absolute numbers of circulating B cells and regulatory T cells (CD25^{hi}CD4⁺) in comparison with a control group of patients with chronic rejection, and a significant decrease in FOXP3 transcript levels in the recipients with chronic rejection (42). Interestingly in this study the blood cell phenotype of clinically tolerant patients did not differ from that of healthy individuals, suggesting that operational tolerance is not due to an increased pool of regulatory T cells but may be due to maintenance of a natural state that is lacking in patients with chronic rejection. By contrast, a different group report a more variable TCR-Vβ repertoire and a higher percentage of CD4⁺CD25^{high} in long-term stable kidney transplant recipients, two of whom were immunosuppression free, in comparison with patients with chronic rejection, dialysis patients and healthy controls (43).

In Brouard et al., 2007 (41) they use a set of 49 genes that gets a maximum sensitivity of 90% in the training set. A set of mostly different 33 genes is said to classify individuals as being tolerant or chronic rejectors. MS4A1, is a molecule also known as CD20. It is expressed in B lymphocytes on the surface. This molecule is present in both gene sets of the Brouard et al., 2007 (41) paper, i.e., the 33 gene and the 49 gene set. Furthermore, Louyet et of, 2005 (44) identify MS4A1 as a marker related to the toleration of grafts in a rat animal model.

It is submitted that there is a need for an improved method for effectively determining an individual's tolerance to an organ transplant.

The present invention provides a method of determining an individual's immunological tolerance to a kidney organ transplantation comprising determining the level of expression of at least two genes selected from the group consisting of TLR5, PNOC, SH2D1B, CD79B, TCL1A, HS3ST1, MS4A1, FCRL1, SLC8A1 and FCRL2 in a sample obtained from the individual.

It has been found that by making the determination set out above it is possible to determine with high specificity and sensitivity whether an individual is immunologically tolerant to the organ transplantation. Specificity is defined as the proportion of true negatives (individuals that are non-tolerant) identified as non-tolerant in the method. Sensitivity is defined as the proportion of true positives (individuals that are tolerant) identified as tolerant in the method. The method provides a highly accurate test that can be performed relatively easily as only a few biomarkers (i.e., the gene expression levels) are measured. A simple and effective test of an individual's tolerance to an organ transplantation is therefore provided.

The term “immunological tolerance” is well known to those skilled in the art and refers to the stable maintenance of good allograft function in the sustained absence of immunosuppressive therapy. In the clinical arena it is only visible when patients experience stable allograft function despite having ceased all immunosuppression for an extended period of time e.g. at least 1 year.

The method of the present invention can be used to determine an individual's tolerance to a kidney transplant.

It has been found that in individuals who are tolerant of a transplanted organ the level of expression of SH2D1B, PNOC, CD79B, TCL1A, HS3ST1, MS4A1, FCRL1 and FCRL2 are raised and that the level of expression of TLR5 and SLC8A1 are reduced.

Any combination of the genes can be used to determine an individual's tolerance to an organ transplant. Although all 10 genes can be used in making such a determination, preferably only 2, 3, 4 or 5 genes are used to make such a determination, more preferably only 3 genes are used to make such a determination.

It is particularly preferred that the method of the present invention comprises determining the level of expression of genes TLR5, PNOC and SH2D1B in a sample obtained from the individual. A positive prediction of an individual's tolerance to an organ transplantation is given by a high level of expression of SH2D1B and PNOC and a low level of expression of TLR5. As will be appreciated by those skilled in the art, the method of the present invention can additionally include determining the expression level of one or more of the following genes CD79B, TCL1A, HS3ST1, MS4A1, FCRL1, SLC8A1 and FCRL2.

The method of the present invention may additionally comprise determining the level of expression of one or more suitable controls. Suitable controls include HPRT, beta-actin and Beta2Microglobulin. The level of the control should not be significantly different between individuals who are tolerant and individuals who are not tolerant.

In a particularly preferred embodiment of the present invention, the probability of an individual being tolerant (P-Tol) is determined by the following formula: P-Tol=e^{Z}/(e^{Z}+1) wherein

Z=−4.4347+2.7191*[SH2DB1]−4.198733*[TLR5]+3.300620*[PNOC]

and a P-Tat score of greater that 0.11 is indicative of an individual being tolerant.

The formula is designed to be applied to gene expression levels determined using microarray analysis. If gene expression levels are determined using other methods, e.g., RT-PCR, the formula may need to be modified. In particular, in a preferred embodiment of the present invention, when the method is performed using RT-PCR the probability of an individual being tolerant (P-Tol) is determined by the following formula: P-Tol=e^{Z}/(e^{Z}+1) wherein

Z=−14.457+94.156*[PNOC]+6.289*[SH2DB1]+5.054*[TLR5]−1.523*[PNOC]*[SH2DB1]−51.584*[PNOC]*[TLR5]−2.339*[SH2DB1]*[TLR5]

and a P-Tol score of greater that 0.0602 is indicative of an individual being tolerant.

The expression of each gene is expressed as 2^{-dCT}, where dCT is calculated as the CT difference between each gene and the control gene.

Other formulae can be used which provide a substantially identical measure of probability. Such alternative formulae will be apparent to those skilled in the art and can be easily calculated.

The method of the present invention can additionally include determining the level of B cells and NK cells. By additionally determining the level of the B cells and the NK the specificity and sensitivity of the method can be further improved. In particular, it has been found that in individuals who are tolerant of a transplanted organ the levels of both the B cells and the NK cells are raised.

The method of the present invention can additionally include determining the level of CD4+CD25^{int} T cells. By additionally determining the level of the CD4+CD25^{int} T cells, the specificity and sensitivity of the method can be further improved. In particular, it has been round that in individuals who are tolerant of a transplanted organ that the level of the CD4+CD25^{int} T cells is reduced relative to total CD4+ T cells.

The method of the present invention can additionally include determining the level of donor specific CD4+ T cells. The level of donor specific CD4+ T cells can be determined using an inteferon gamma ELISPot assay as described below. By additionally determining the level of donor specific CD4+ T cells, the specificity and sensitivity of the method can be further improved. By measuring the level of donor specific CD4+ T cells, the response of the individual to the donor organ can be determined. In particular, it has been found that in individuals who are tolerant of a transplanted organ that the level of such a response the level of the donor specific CD4+ T cells) is reduced.

The method of the present invention can additionally include determining the ratio of FoxP3 to α-1,2-mannosidase gene expression level of CD4+ T cells. By additionally determining the ratio of FoxP3 to α-1,2-mannosidase gene expression level of CD4+ T cells the specificity and sensitivity of the method can be further improved. In particular, it has been found that in individuals who are tolerant of a transplanted organ that the ratio is increased.

The method of the present invention can additionally include determining the ratio of CD19+ to CD3+ cells. By additionally determining the ratio of CD19+ to CD3+ cells the specificity and sensitivity of the method can be further improved. In particular, it has been found that in individuals who are tolerant of a transplanted organ that the ratio is increased.

The method is performed on a sample obtained from the individual. The sample may be any suitable sample from which it is possible to measure the markers mentioned above. Preferably the sample is blood, Serum or other blood fractions, urine or a graft biopsy sample. Most preferably the sample is a peripheral blood sample.

SH2D1B (SH2 domain containing protein 1B) is a standard term well known to those skilled in the art. In particular, the sequences of the polymorphic human forms of SH2D1B are given in the NCBI protein database under accession number GI:42744572, version AAH66595.1; accession number GI:54792745, version NP_{—}444512.2; accession number GI:18490409, version AAH22407.1; and accession number GI:559613297, version CAI15780.1.

TLR5 (Toll-like receptor 5 protein) is a standard term well known to those skilled in the art. In particular, a few exemplary sequences of the polymorphic human forms of TLR5 given in the NCBI protein database are under accession number GI:80478954, version AAI09119.1; accession number GI:80475052, version AAI09120.1; accession number GI:13810568, version BAB43955.1; and accession number GI:222875780, version ACM69034.1.

PNOC (Nociceptin) is a standard term well known to those skilled in the art. In particular, the sequences of the polymorphic human forms of PNOC are given in the NCBI protein database under accession number GI:49456885, version CAG46763.1; and accession number GI:49456835 version CAG46738.1

CD79B (B-cell antigen receptor complex-associated protein beta-chain) is a standard term well known to those skilled in the art. In particular, the sequences of the polymorphic human forms of CD79B are given in the NCBI protein database under accession number. GI:1087009, version. AAC60654.1; and accession number GI:20987620, version AAH30210.1.

TCL1A (T-cell leukemia/lymphoma 1A) is a standard term well known to those skilled in the art. In particular, the sequences of the polymorphic human forms of TCL1A are given in the NCBI protein database under accession number GI:48145709, version CAG33077.1; accession number GI:148922879, version NP_{—}001092195.1; accession number GI:11415028, version NP_{—}068801.1; accession number GI:13097750, version AAH03574.1; accession number GI:46255821, version AAH14024.1: and accession number GI:13543334, version AAH05831.1.

HS3ST1 (Heparan sulfate (glucosamine) 3-O-sulfotransferase 1) is a standard term well known to those skilled in the art. In particular, the sequences of the polymorphic human forms of HS3ST1 are given in the NCBI protein database under accession number GI:116283706, version AAH25735.1; and accession number GI:34785943, version AAH57803.1.

MS4A1 (Membrane-spanning 4-domains, subfamily A, member 1; B-lymphocyte antigen CD20) is a standard term well known to those skilled in the art. In particular, the sequences of the polymorphic human forms of MS4A1 are given in the NCBI protein database under accession number GI:23110989, version NP_{—}690605.1; accession number GI:23110987, version NP_{—}068769.2; and accession number GI:12803921, version AAH02807.1.

FCRL1 (also referred to as THC2438936 herein) (Near 3′ of Fc receptor-like protein 1 (FCRL1) gene) is a standard term well known to those skilled in the art. In particular, a few exemplary sequences of the polymorphic human forms of FCRL1 given in the NCBI protein database are under accession number GI:55662454, version. CAH73053.1; accession number GI:55661513, version CAH70234.1; accession number GI:55661511, version CAH70232.1; accession number GI:21707303, version AAH33690.1; and accession number GI:117606520, version ABK41917.1.

SLC8A1 (solute carrier family 8 (sodium/calcium exchanger), member 1) is a standard term well known to those skilled in the art. In particular, the sequences of the polymorphic human forms of SLC8A1 are given in the NCBI protein database under accession number GI:68087008, version AAH98285.1; and accession number GI:67514242, version AAH98308.1.

FCRL2 (Fc receptor-like protein 2) is a standard term well known to those skilled in the art. In particular, a few exemplary sequences of the polymorphic human forms of FCRL1 given in the NCBI protein database are under accession number GI:55662464, version CAH73063.1; accession number GI:55662461, version CAH73060.1; accession number GI:46623042, version AAH69185.1; and accession number GI:117606518, version ABK41916.1.

FoxP3 (forkhead box P3) is a standard term well known to those skilled in the art. In particular, the sequences of the polymorphic human forms of FoxP3 are given in the NCBI protein database under accession number GI:146262391, version number ABQ15210.1; accession number GI:219518921, version AAI43787.1; accession number GI:219517996, version AAI43786.1; accession number GI:109731678, version AAI13404.1, accession number GI:109730459, version AAI13402.1; and accession number GI:63028441, version AAY27088.1.

1,2-alpha mannosidase is a standard term well known to those skilled in the art. In particular, the term refers to the 1,2-alpha mannosidase A1 form. Sequence of the human form of 1,2-alpha mannosidase A1 is given in the NCBI protein database under accession number GI:24497519, version number NP_{—}005898.2.

For the avoidance of doubt the specific sequences of the markers mentioned above are defined with respect to the version present, in the database at the priority date of the present application. The specific sequences of the markers are exemplary. Those skilled in the art will appreciate that polymorphic variants exist in the human population.

There are numerous ways of determining the level of expression of the genes, including Northern blotting, mRNA microarrays. RT-PCR methods, differential display. RNA interference, reporter gene assays and tag based technologies like serial analysis of gene expression (SAGE). Such methods are well known to those skilled in the art (see for example Measuring Gene Expression by Matthew Avison, 2007, published by Taylor & Francis Group; ISBN: 978-0-415-37472-9 (paperback) 978-0-203-88987-9 (electronic)). Levels of the encoded protein expressed can also be measured to determine the level of gene expression. Numerous methods of determining the level of protein expression are well know to those skilled in the art.

The levels of the various cell types that can be measured in the present methods as additional biomarkers can be detected using any suitable method. For example, flow cytometry using appropriate antibodies can be used. Such methods are well known to those skilled in the art.

The level of donor specific hyporesponsiveness of CD4+ T cells can be determined using any suitable method. Suitable methods include measuring IFNgamma production by ELISA, Luminex methods or by intracellular cytokine production using flow cytometry. In making such measurements, it is preferred that the method comprises the following steps;
a. Having a set number of CD4+ T cells from the recipient;
b. Stimulating the CD4+ T cells with cells from the donor or cells from an individual that has the same HLA-class II as the donor (at serological precision), wherein the cells have been irradiated (preferably the cells are PBMC that have been depleted of T and NK cells (using CD2 and TCRgd antibodies);
c. Stimulating the CD4+ T cells with cells from a “3^{rd} party” that has similar HLA-class-II mismatches as those present between donor and recipient (preferably the cells are PBMC that have been depleted of T and NK cells (using CD2 and TCRgd antibodies);
d. Stimulating the CD4+ T cells with cells from a “4^{th} party” that has complete HLA-class-II mismatch to both the donor and the recipient (preferably the cells are PBMC that have been depleted of T and NK cells (using CD2 and TCRgd antibodies); and
e. determining the relative levels of IFNgamma production by the CD4+ T cells.

A suitable method for determining the level of donor specific CD4+ T cells is described herein below.

In order to determine whether the level of the markers referred to above is greater than (high) or less than (low) normal, the normal level of a relevant population of non-tolerant individuals is typically determined. The relevant population can be defined based on, for example, organ transplanted, level and type of immunosuppressive medication, ethnic background or any other characteristic that can affect normal levels of the markers. Once the normal levels are known, the measured levels can be compared and the significance of the difference determined using standard statistical methods. If there is a substantial difference between the measured level and the normal level (i.e. a statistically significant difference), then the individual from whom the levels have been measured may be considered to be immunologically tolerant.

The technology described herein allows the monitoring of an individual's tolerance to the graft (i.e. transplanted organ) and thereby can identify individuals that can stop taking immunosupression medication or reduce the level of immunosupression medication. The present technology may also assist with the management of immunosupression protocols and the post-transplantation management of transplant organ recipients.

The present invention also provides a sensor for detecting the expression levels of at least 2 genes selected from the group consisting of TLR5, PNOC, SH2D1B, CD79B, TCL1A, HS3ST1, MS4A1, FCRL1, SLC8A1 and FCRL2. Preferably the sensor is for detecting the expression levels of the TLR5, PNOC and SH2D1B genes. Suitable sensors for monitoring the expression levels of genes in a microarray are well know to those skilled in the art and include mRNA chips, protein expression sensor, etc. The sensors generally comprises one or more nucleic acid probes specific for the gene being detected adhered to the sensor surface. The nucleic acid probe thereby enables the detection of a gene transcript from the target gene. Preferably the sensor is additionally for detecting the expression of one or more, preferably all, of the following genes CD79B, TCL1A, HS3ST1, MS4A1, FCRL1, SLC8A 1 and FCRL2.

The present invention also provides a kit comprising reagents for detecting the level of expression of at least 2 genes selected from the group consisting of TLR5, PNOC, SH2D1B, CD79B, TCL1A, HS3ST1, MS4A1, FCRL1, SLC8A1 and FCRL2. Preferably the kit comprises reagents for detecting the level of expression of the TLR5, PNOC and SH2D1B genes. Preferably the kit further comprises reagents for detecting the level of expression of one or more of the following genes CD79B, TCL1A, HS3 ST1, MS4A1, FCRL1, SLC8A1 and FCRL2. The reagents for detecting the level of expression of the genes are preferably reagents for detecting the level of gene expression of the genes by RT-PCR.

The kit can also include a computer programmed with an algorithm for calculating the individual's probability of being tolerant, instructions and other items useful for performing the method described herein.

Particular aspects of this technology are described by way of example, below with reference to the following figures.

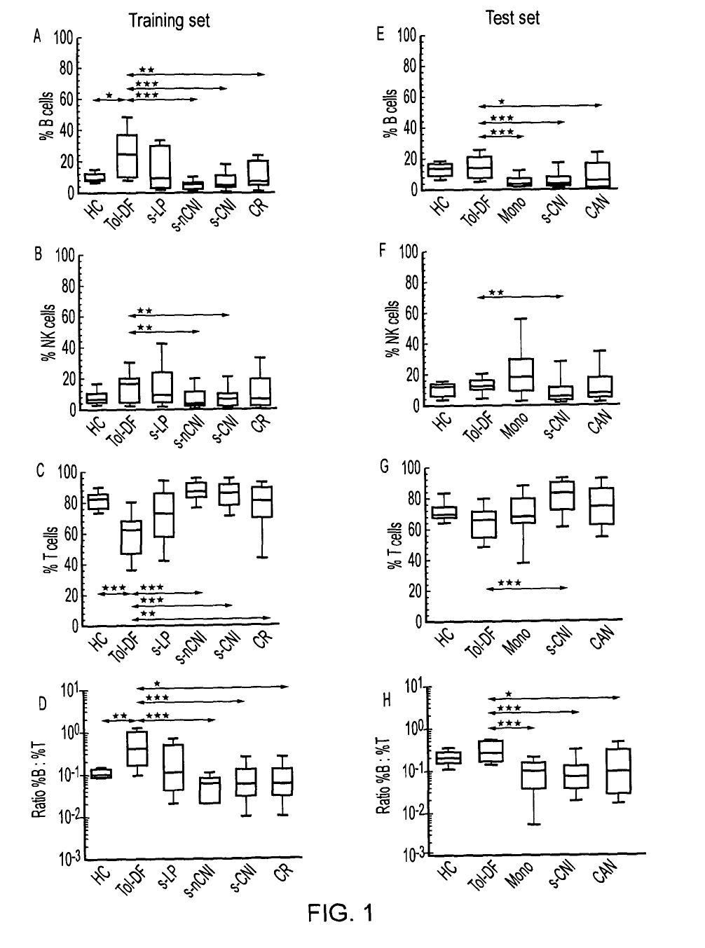

FIG. 1 shows the flow cytometry analysis of peripheral blood lymphocyte subsets of the training (A-D) and test set (E-H). Lymphocyte subsets were defined as: B cells as CD19+ lymphocytes (A,E), NK cells as CD56+CD3− lymphocytes (B,F); T cells as CD3+ lymphocytes (C,G). Ratio of CD19+:CD3+ is shown (D,H). Median and interquartile range are shown. Two-sided p values for Mann-Whitney U test comparisons between Tol-DF patients and other groups are shown (*** p<0.001, ** p<0.01 or * p<0.05). P values for comparisons between other study groups are shown in Table 5.

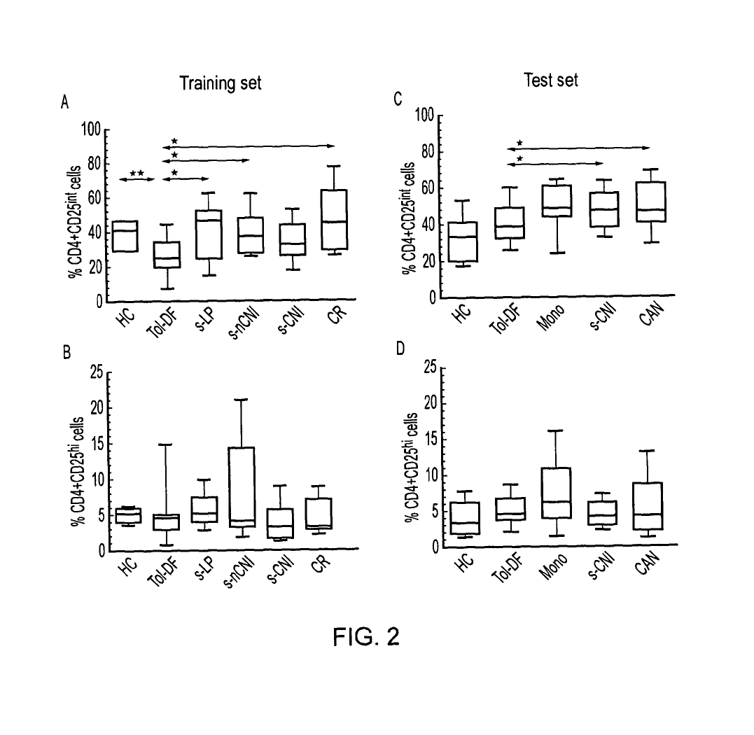

FIG. 2 shows the flow cytometry analysis of peripheral blood T cell expression of CD25 of the training (A & B) and test sets (C & D). Median and interquartile range of the percentages of CD4+ T cells with intermediate (CD4+CD25int) and high (CD4+CD25hi) CD25 expression are shown. Two-sided p values for Mann-Whitney U test comparisons between Tol-DF patients and the rest of the groups are shown (**) p<0.01 or (*) p<0.05. P values for comparisons between other study groups are shown in Table 5.

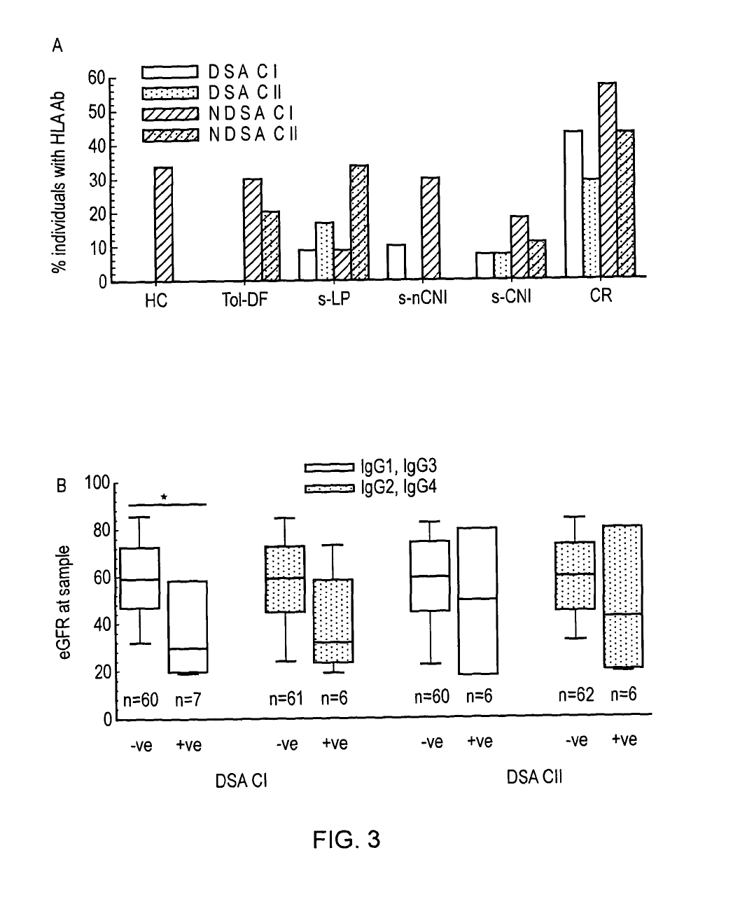

FIG. 3 shows (A) Percentage of patients per group with positive detection of serum donor specific (DSA) and non specific (NDSA) anti-HLA class I (CI) and class II (CII) antibodies in the training set. (B) Renal function of patients in whom complement-fixing (IgG1, IgG3) or non-complement-fixing (IgG2, IgG4) DSA were present (+ve) or absent (−ve). Median and interquartile range is shown. Two-sided p values for Mann-Whitney U test comparisons between groups are displayed (* p<0.05). Of note, DSA levels were absent in tolerant recipients.

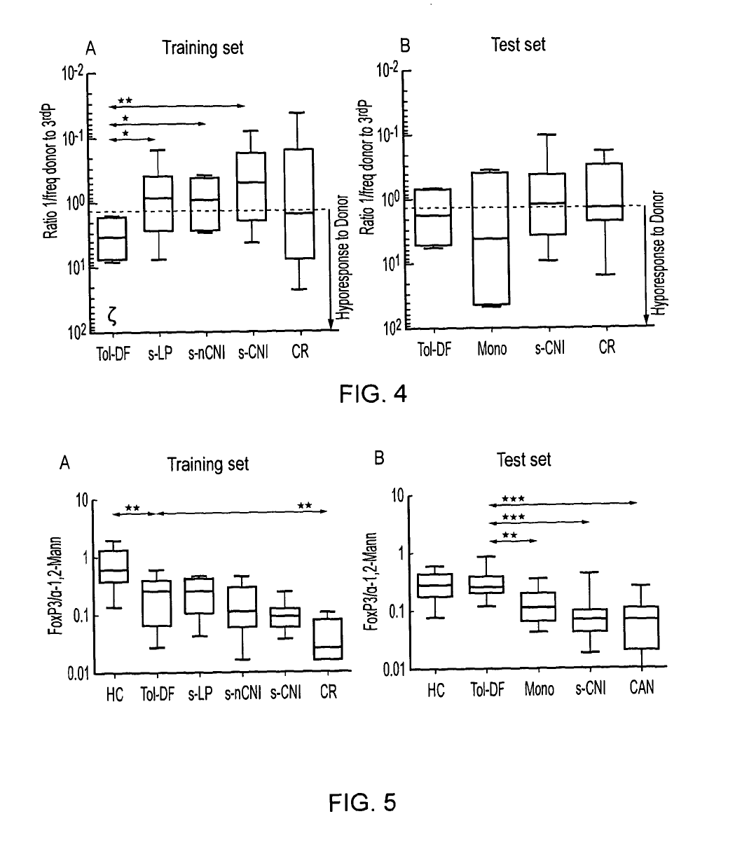

FIG. 4 shows the IFNγ ELISpots used to detect direct pathway alloresponses in patients of the (A) training and (B) test set. The number of IFNγ producing cells in recipient CD4+ T cells was calculated (background-deducted) when stimulated with donor cells and third party cells (3rdP), to obtain a frequency of responder cells. Median and interquartile ranges for the ratio of responder frequencies on donor:3rdP stimulation are shown. Ratio values>1.5 indicate hyporesponsiveness to donor. Two-sided p values for Mann-Whitney U test comparisons between groups are shown (** p<0.01, * p<0.05). Individual patient IFNγ ELISpot responder frequencies to donor and 3rdP are shown in FIG. 10. ζ: Wilcoxon test between donor and 3rdP frequencies p<0.05.

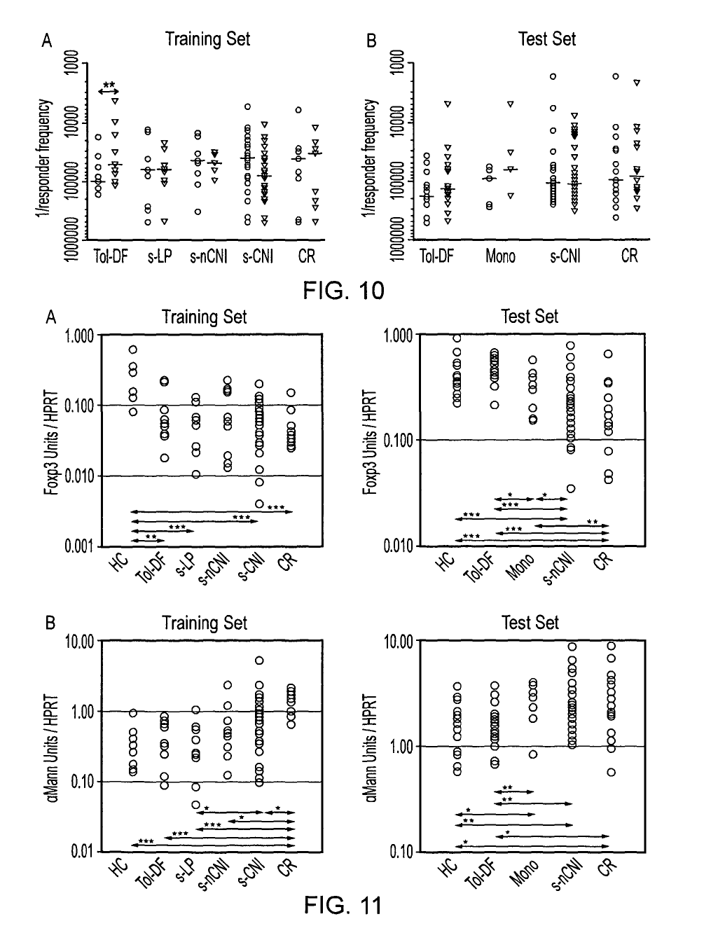

FIG. 5 shows the qRT-PCR gene expression analysis of peripheral blood expression of FoxP3 and α-1,2-mannosidase. A ratio of the expression values of FoxP3 and α-1,2-mannosidase was calculated patients of the training set (A) and test set (B). Median and interquartile range is shown. Two-sided p values for Mann-Whitney U test comparisons between Tol-DF and other groups are shown (*** p<0.001, ** p<0.01). Statistical values for comparisons between other study groups are shown in Table 6. Individual expression values for FoxP3 and α-1,2-mannosidase are also shown in FIGS. 11A and 11B, respectively.

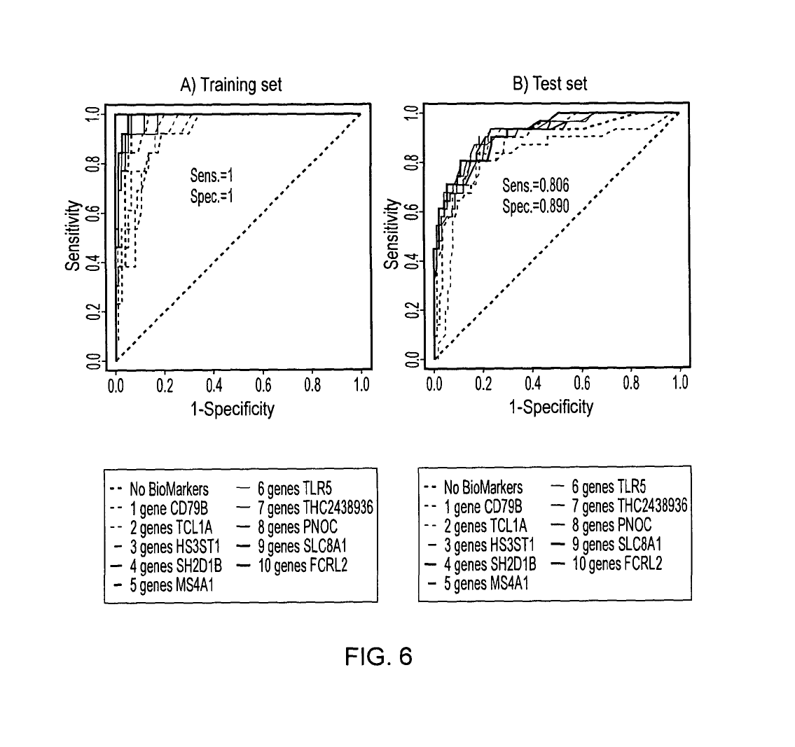

FIG. 6 shows the ROC curves of the training (A) and test set (B) generated using up to 10 highest ranked genes (black lines). Significant differential gene expression was detected by microarray analysis of peripheral blood. Using a binary regression model for classification ROC curves (Tol-DF vs non-tolerant groups, excluding HC) were generated using the top 10 ranked significant genes identified by four-class Kruskal-Wallis analysis of microarray data. Genes were ranked within the training set based on their p value with 1% FDR. The same 2-class model was used to assess the diagnostic capabilities of the same genes to detect Tol-DF recipients within the test set.

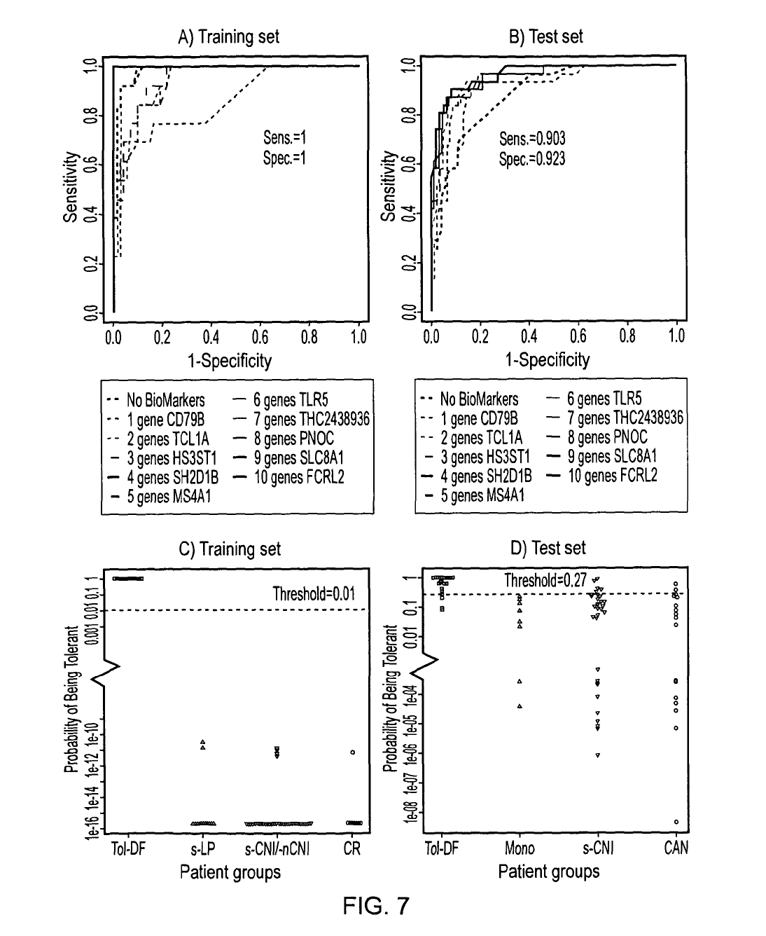

FIG. 7 shows the ROC curves of the training set (A) and test set (B) generated using crossplatform biomarkers and genes identified by microarray analysis. Two-class ROC curves (Tol-DF vs non-tolerant groups, excluding HC) were generated using 4 biomarkers: B/T lymphocyte ratio, % CD4+CD25int, ratio of anti-donor:anti-3rdP ELISpot frequencies and ratio of FoxP3/α-1,2-mannosidase expression, combined with sequential addition of 10 most significant genes. Estimated probabilities of patients from each study group of the training set (C) and test set (D) of being classified as tolerant based on the cross-platform biomarker signature of tolerance (4 biomarkers+10 genes) was calculated using a binary regression procedure.

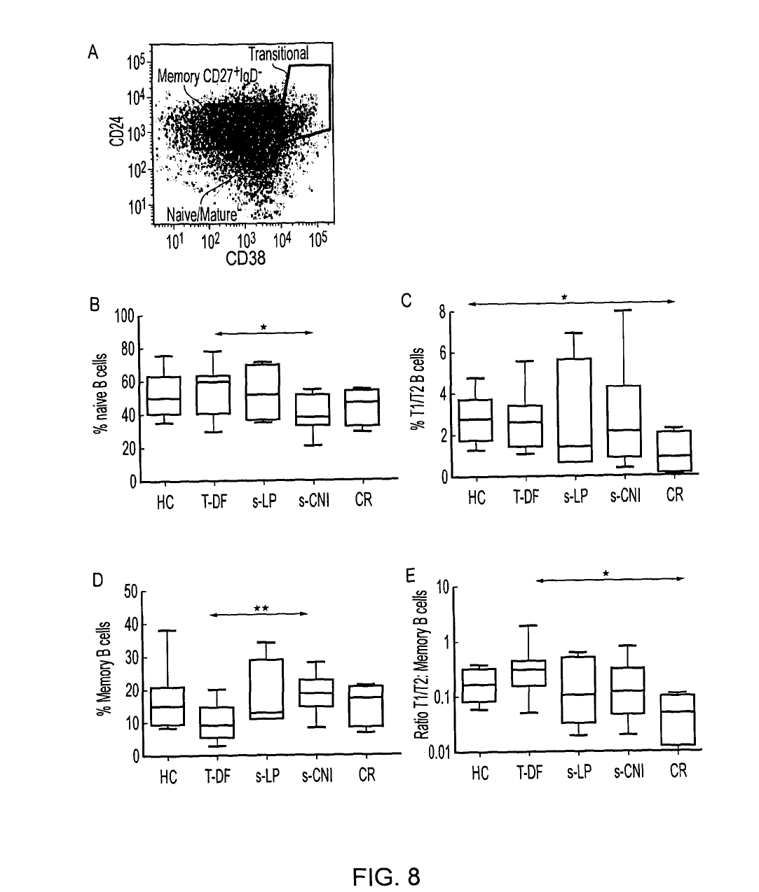

FIG. 8 shows the analysis of peripheral blood B cell subsets in training and test set patients. (A) B cell subsets were analysed by gating on CD9+ lymphocytes and defined as follows: late-memory B cells CD19+CD27+IgD−CD24+CD38−/int; naïve/mature B cells CD19+CD27−CD24intCD38int; T1/T2 transitional B cells CD19+CD27−CD24+CD38hi. Percentages of naïve B cells (B), T1/T2 transitional cells (C) and memory B cells (D) of total B cells. Ratio of the percentage of T1/T2 transitional: memory B cells (E). Median and interquartile range is shown. When significant, p values for 2-tailed Mann-Whitney U test comparisons between groups are shown (* p<0.05, ** p<0.01).

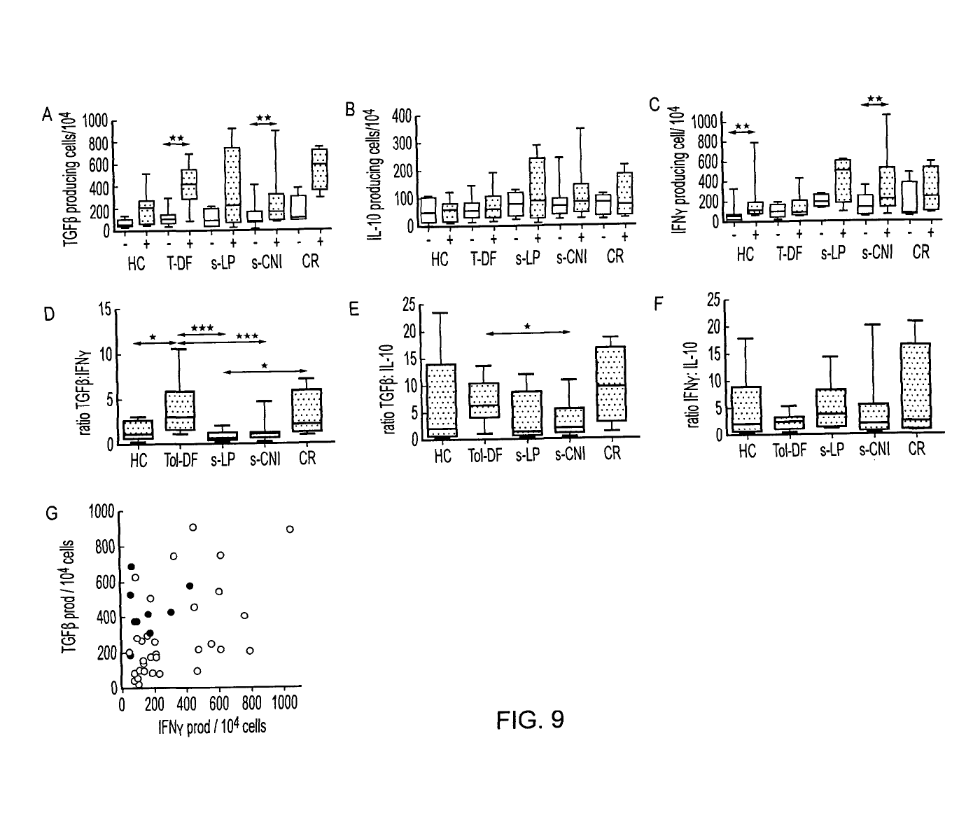

FIG. 9 shows the analysis of B cell cytokine production. B cell production of (A) TGFβ, (B) IL-10 and (C) IFNγ was assessed by intracellular cytokine staining after in vitro stimulation of PBMC with phorbol 12-myristate 13-acetate and ionomycin. Results are expressed as the number of cytokine producing B cells (gated CD19+ lymphocytes) detected per 1×10⁴ B cells analysed by flow cytometry in stimulated (+) and unstimulated (−) cultures, with median and interquartile range shown for each group. Figures (D-F) show the number of cytokine producing cells per 1×10⁴ stimulated B cells expressed as a ratio of each cytokine response. Figure G shows TGFβ and IFNγ cell cytokine responses, where each patient is represented by a filled circle and black filled circles represent Tol-DF patients, showing that B cells of Tol-DF patients have a higher capacity to produce TGFβ rather than IFNγ. P values for Wilcoxon-matched pair test (A-C) and 2-tailed Mann-Whitney U test comparisons between groups (D-F) are shown (*p<0.05, **p<0.01, ***p<0.001). Sample n: HC=8, Tol-DF=10, s-LP=8, s-CNI=16, CR=4.

FIG. 10 shows the cellular functional assays detecting direct pathway alloresponses by IFNγ ELISpot in the (A) training and (B) test sets. The number of IFNγ producing cells in recipient CD4+ T cells was calculated. Data shows the frequency (1 responder/n cells) and median group frequency (black bar) of T cell responses to donor APCs (∘ anti-Donor response) or equally mismatched 3rd Party APCs (an inverted triangle indicates an anti-3rdP response) after deducting background. Two-sided p values for Wilcoxon matched paired test (** p<0.01) between donor and 3rdP frequencies is shown.

FIG. 11 shows qRT-PCR analysis of (A) FoxP3 and (B) α-1,2-mannosidase expression in whole peripheral blood samples of the training and test sets. Expression levels of FoxP3 and α-1,2-mannosidase are expressed as units relative to the expression of HPRT. When significant, p values for 2-tailed Mann-Whitney U test comparisons between, groups are shown (* p<0.05, ** p<0.01, *** p<0.001).

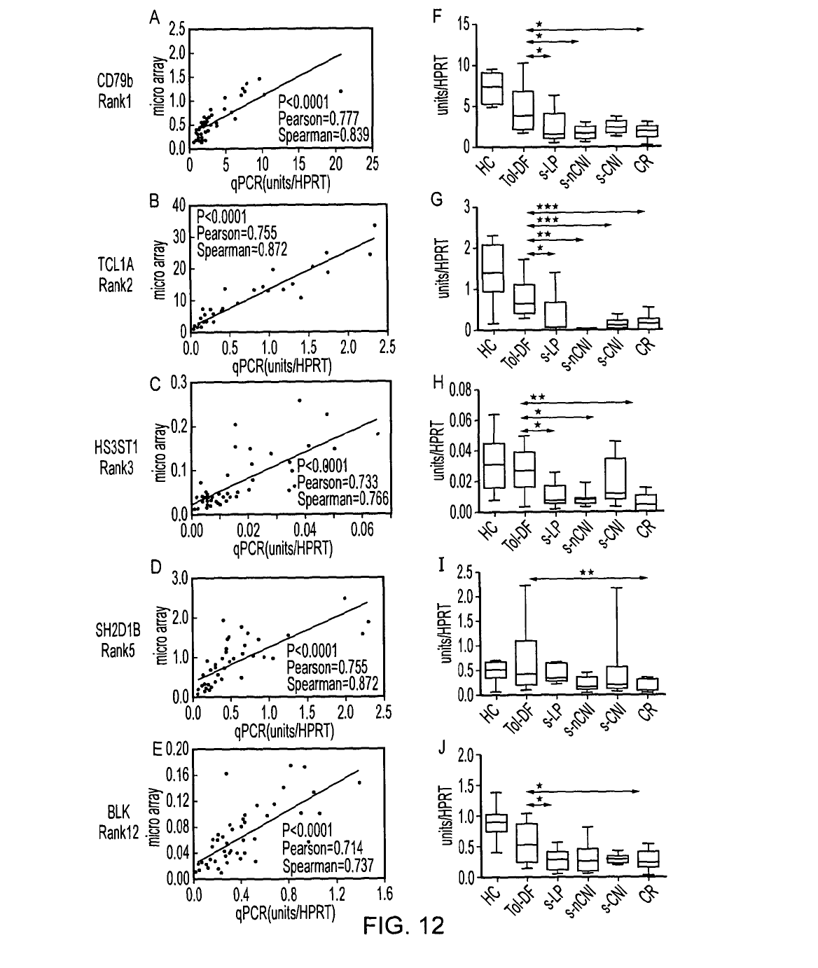

FIG. 12 shows the correlation of gene expression by microarray and qRT-PCR for selected genes that displayed significantly differential expression (A-E) for training set. Signal intensity data of each probe on the microarray was calculated for each patient and expression relative to HPRT was then calculated for each gene as log 2 [gene of interest]-log 2 [HPRT]. qRT-PCR data are depicted as units/HPRT. Pearson and Spearman rank correlation coefficients and p-values are shown. RT-PCR gene expression data for the same set of genes is shown in F-J). Median and interquartile range for each group is shown. Statistical comparison between groups was assessed by Mann-Whitney U test and significant p values are shown (* p<0.05, ** p<0.01, *** p<0.001).

FIG. 13 shows the correlation of gene expression by microarray and qRT-PCR for selected genes that displayed significantly differential expression (A-E) for test set. Signal intensity data of each probe on the microarray was calculated for each patient and expression relative to HPRT was then calculated for each gene as log 2 [gene of interest]-log 2 [HPRT]. qRT-PCR data are depicted as units/HPRT. Pearson and Spearman rank correlation coefficients and p-values are shown. RTPCR gene expression data for the same set of genes is shown in F-J). Median and interquartile range for each group is shown. Statistical comparison between groups was assessed by Mann-Whitney U test and significant p values are shown (* p<0.05, ** p<0.01, *** p<0.001).

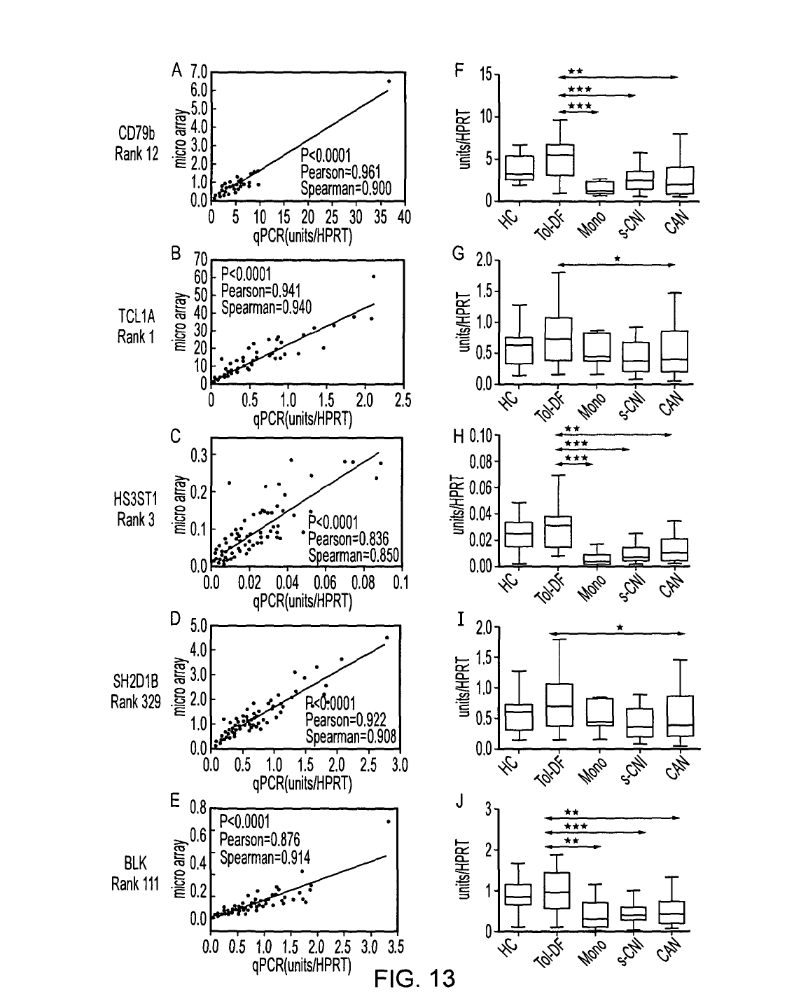

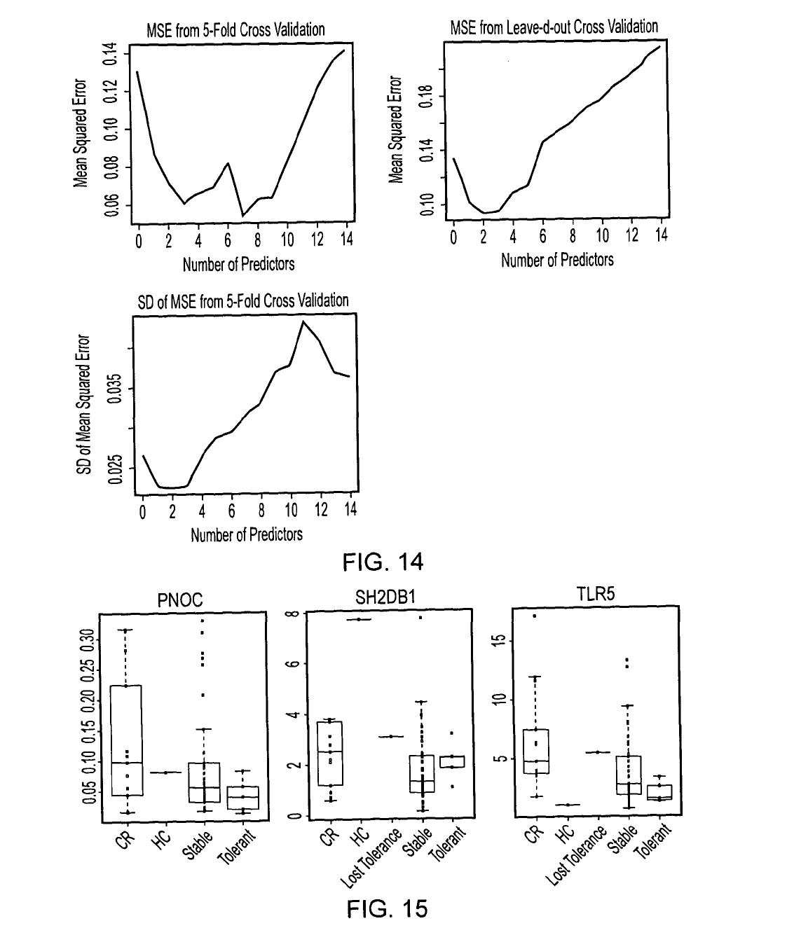

FIG. 14 shows the results of a binary regression model used to cross validate selected combinations of biomarkers. MSE=Mean Squared Error, SD=Standard Deviation.

FIG. 15 is a Boxplot showing expression levels of the 3 genes (SH2DB1, PNOC and TLR5) per group.

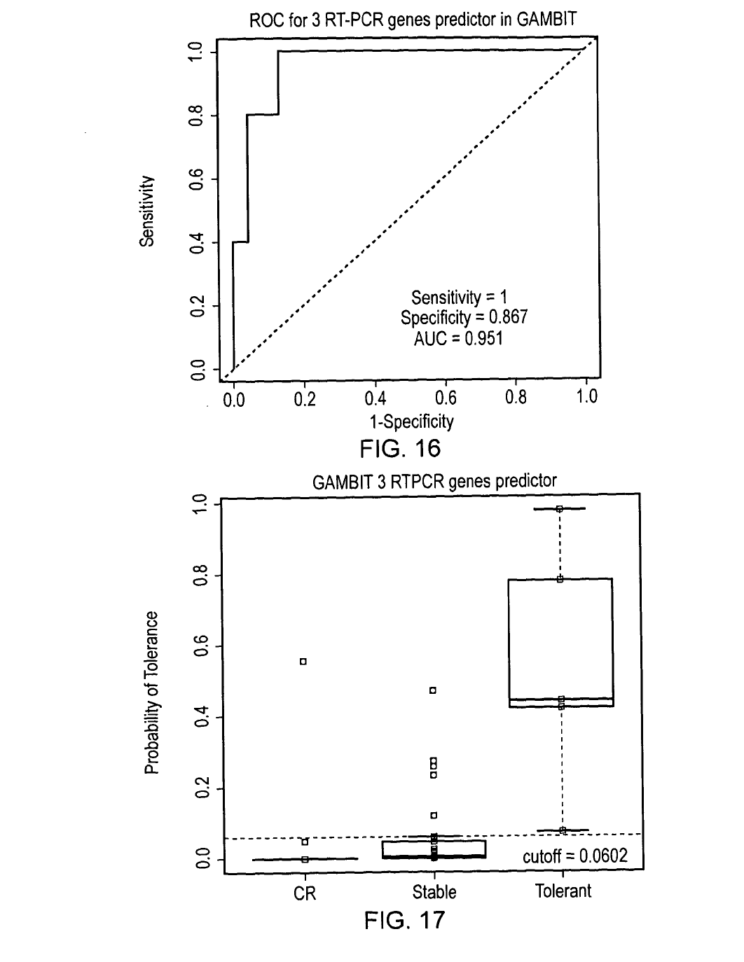

FIG. 16 is ROC curve obtained using a Logistic Regression classifier with the main and interaction effects between the three genes TLR5, PNOC and SH2DB1. AUC, Sensitivity and Specificity are calculated for the optimal cutoff in this sample (i.e. 0.0602).

FIG. 17 shows Boxplots of Probability of Tolerance estimated by logistic regression classifier using the 3 genes (SH2DB1, PNOC and TLR5).

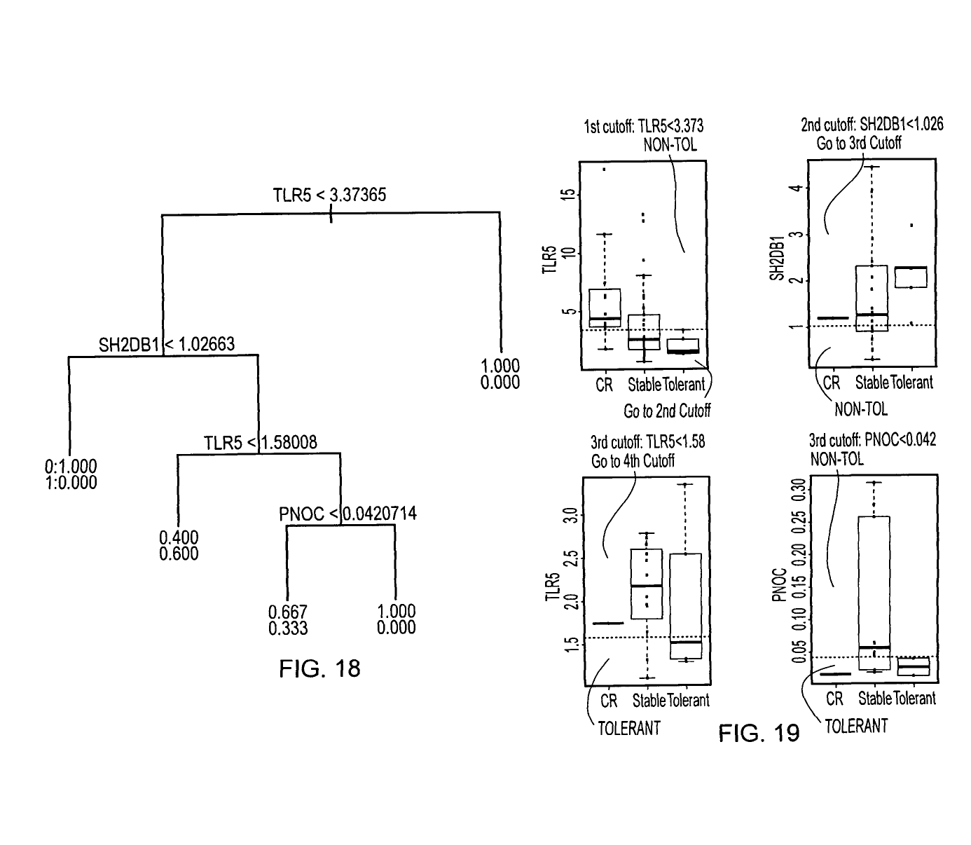

FIG. 18 shows a classification cation tree estimated with 3 genes, TLR5, SH2DB1 and PNOC. The Figure shows cutoffs at each split and probability of each outcome assigned at each terminal node (0—above-being non-tolerant, and 1—below-being tolerant).

FIG. 19 shows a series of Boxplots illustrating the application of the tree's cutoffs.

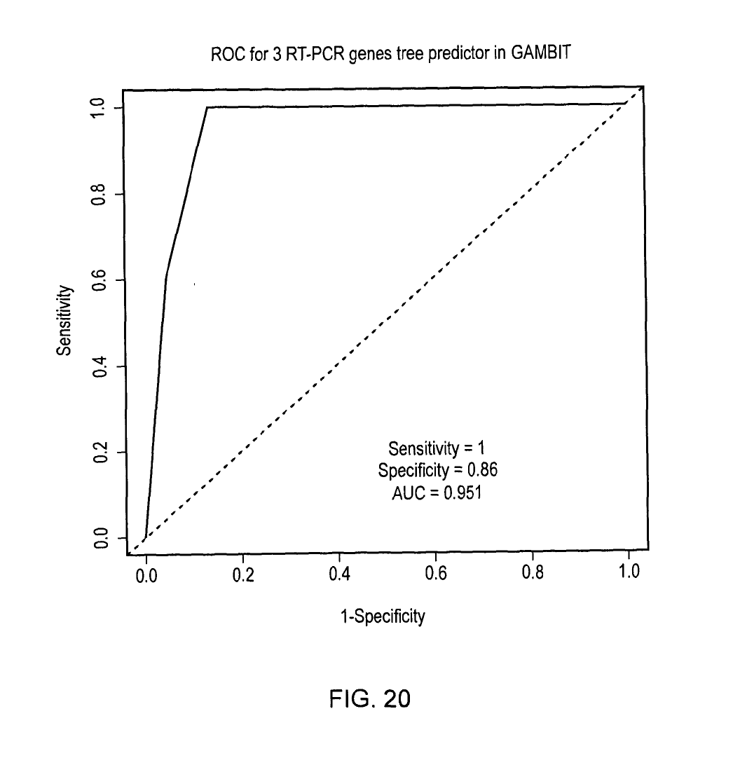

FIG. 20 shows an ROC curve resulting from the use of the estimated classification tree with 3 genes to predict tolerance.

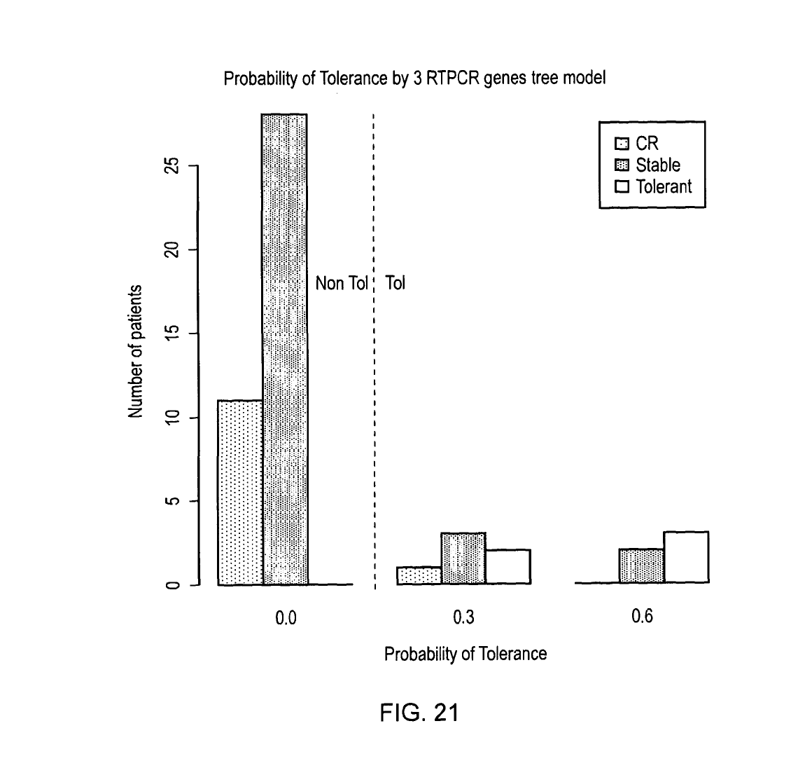

FIG. 21 shows a barplot with frequencies of probability of tolerance assigned by the classification tree by patient group.

### EXAMPLES

### Materials and Methods

### Study Patients:

### Training Set Description:

This cohort of patients recruited by the Indices of Tolerance network (IOT) consisted of 71 kidney transplant recipients and 19 age and sex matched healthy controls (HC). Five patient groups were included: eleven functionally stable kidney transplant recipients (serum creatinine (CRT)<160 μmol/l and <10% rise in the last 12 months) despite having stopped all their immunosuppression for more than one year (Tol-DF), eleven patients with stable renal function (same criteria) maintained on less than 10 mg/day of prednisone as the only immunosuppressive agent (s-LP), ten patients maintained on immunosuppression (Azatbioprine and Prednisone) in the absence of a calcineurin inhibitor since transplantation (s-nCNI), thirty patients maintained on standard calcineurin-inhibitor therapy (s-CNI), nine patients with biopsy proven (all reevaluated for this study) and immunologically driven chronic rejection (CR). Patient clinical characteristics are described in Table 1. All samples were processed and analysed in a blinded fashion.

### Test Set Description:

An independent set of kidney transplant recipients were recruited in the USA (the ITN cohort). The ITN cohort consisted of (1) “Tol-DF” (n=24) functionally stable kidney transplant recipients (serum creatinine (CRT) within 25% of baseline) despite having stopped all their immunosuppression for more than one year; (2) “Mono” (n=11) patients with stable renal function that are maintained on monotherapy with steroids; (3) “s-CNI” subjects (n=34), with clinically stable renal function using the same criteria as Tol-DF while on maintenance with a triple drug immunosuppressive regimen (including a calcineurin or mTOR inhibitor, an anti-proliferative agent and corticosteroids) and “CAN” participants (n=20), defined as those with chronic allograft nephropathy and impaired renal function (50% increase in their baseline CRT at time of enrolment relative to their initial post-transplant baseline) due to presumed immune mediated allograft rejection. An additional group of 31 healthy control volunteers (HC) with no known history of renal disease/dysfunction or evidence of acute medical illness was enrolled. Group characteristics are summarised in Table 3. Whole blood mRNA and frozen PBMC were received by labs performing the selected validation assays described.

### Blood Samples:

The training set samples were processed in all cases within 24 hours of venesection. PBMCs were obtained by density gradient centrifugation using Lymphocyte Separating Medium (PAA Laboratories, Somerset UK). Cells were washed and resuspended in 10% DMSO (Sigma, Dorset UK) and human serum (Biowest, France) and frozen immediately at −80° C. After 24 hrs cells were transferred into liquid nitrogen and kept until use.

### Flow Cytometry on PBMC:

Thawed PBMC were washed and resuspended at 1×10⁶/mL. Titrated amounts of fluorochrome-conjugated monoclonal antibodies were used to identify leucocytes, CD45+CD14− for lymphocytes. CD3+ for T cells, CD19+ for B cells, CD56+CD3− for NK cells, CD4+CD3+ for CD4 T cells, CD8+CD3+ for CD8 T cells. B cell subsets were defined as previously described (30), as CD19+CD27+IgD−CD2+CD38−/int for late-memory B cells, CD19+CD27−CD24intCD38int for naïve/mature B cells and CD19+CD27=CD24+CD38hi for T1/T2 transitional B cells (All from Caltag, Burlingham USA). Cells were fixed with 1% paraformaldehyde/PBS and data acquired on a FACScalibur within 48 hours. CD25 expression was studied on CD4+ T cells as described in (31). B cell production of TGFβ (from R&D systems), IL-10 and IFNγ (both from eBioscience UK) was assessed by intracellular cytokine staining on in vitro stimulated PBMC with 500 ng/mL phorbol 12-myristate 13-acetate and ionomycin in presence of 2 μM monensin and 10 μg/mL brefeldin-A for 5 hours at 37° C. A minimum of 10,000 CD19+ events were acquired for each sample.

### Anti-Donor Antibody Detection:

Peripheral blood was collected in clotting activator vacutainers (Becton Dickinson, San Jose USA) and allowed to clot for a minimum of 2 and a maximum of 24 hours. Samples were centrifuged and collected serum stored at −80° C. until use.

Screening for IgG anti-HLA antibodies of any specificity by xMAP® (Luminex) technology (32). After washing, HLA-coated Luminex screening beads and 12.5 μl of patient serum or control serum were added on a plate and mixed gently for 30 minutes in the dark. Plates were washed three times and PE-conjugated goat anti-human IgG (1:10) added to each test well. Plates were incubated for 1 hour, wash buffer added and then data collected using the Luminex100 instrument, as recommended by the manufacturers.

### Screening for IgG Subclass and Anti-HLA Broad Specificity:

Positive sera were tested for IgG subclass identification and class I and class II broad specificity distinction. Screening was performed using class I and II Luminex identification kits (Quest Biomedical). Secondary antibodies used for detection of bound patient antibodies were as follows: anti-human IgG1 conjugated to biotin (clone 8c/6-39, Sigma) anti-human IgG2 conjugated to biotin (clone HP-6014, Sigma), anti-human IgG3 conjugated to biotin (clone HP-6050, Sigma), anti-human IgG4 conjugated to biotin (clone HP-6050, Sigma), and streptavidin-phycoerythrin (Calbiochem).

### Cell Fractions for Functional Assays:

PBMCs were thawed on the day of the assay. T cell subsets CD4+ and CD4+CD25−(CD4+ depleted of CD25+ cells) were separated using standard methods of negative immune-isolation as previously described (33). Purity was verified by flow cytometry. In particular, the inventors have used two specific sets of monoclonal antibodies with a fluorochrome bound to stain isolated peripheral blood mononuclear cells. The following analysis was used on lymphocytes (selected by forward side and on CD45+CD14− expression). The first set included: TCR gamma/delta-FITC, CD25-PE, CD4-APC. The level of CD4+CD25^{int} was obtained selecting the CD4+ T cells and within this subset studying the intermediate expression of CD25 (defined from CD25negative to CD25 as high as CD4NEG cells showed, CD25 high cells are excluded from this gate). The second set included: CD3-FITC, CD56-PE and CD19-APC. The variable “B.T” was obtained selecting the CD19+ cells and dividing that percentage by the percentage of CD3+ lymphocytes.

### Donor, Surrogate Donor and 3^{rd} Party Cells:

Cells front the 31 living kidney donors were used for the 71 donor-specific cellular assays on the training set, and 28 donors for 64 cell samples on the test set. Where donor blood was unavailable, surrogate donor cells were obtained that had equal HLA class II expression as the original donor. These cells and similarly mismatched 3^{rd} party cells were used from: healthy volunteers from the Anthony Nolan bone marrow registry, HLA-typed healthy volunteers and splenocytes collected at the time of cadaveric donation at the Hammersmith and Guy's Hospitals in London. Similarly mismatched 3^{rd} party cells were selected by the number of HLA mismatches for class II (HLA-DR and HLA-DQ) when compared to the relevant donor and recipient.

### MLR Cultures for ELISpot:

Human IFNγ-ELISpotPRO (Mabtech, Sweden) kits were used and developed according to manufacturer's instructions. Background deducted positive spots were enumerated using an automatic image analyser for ELISpot plates (AID, Germany). Quantitative assessment of direct pathway donor antigen-specific responder T cell frequencies was made by stimulating recipient CD4+ T cells with T cell- and NK cell-depleted PBMCs (APCs) separated from either donor PBMCs or HLA-typed 3^{rd} party cells. Allogeneic MLR cultures were performed over 24 hours. Duplicates were set up with three doubling dilutions starting typically at 2×10⁵ responder cells per well. The ratio of stimulator to responder cells was kept constant by always using half the number of APCs compared to the number of responder cells used in the top dilution, typically 1×10⁵ responders per well. Donor reactivity was expressed as a ratio of frequency to donor and frequency to 3^{rd} party. The inverse of the frequency was recorded in the database (i.e. 1 in 54,000 cells was recorded as 54,000), therefore ratio values>1.5 were defined as indicating a hyporesponse to donor stimulation.

### Blood Sampling for Gene Expression Analysis:

For the training set cohort peripheral vein blood was drawn directly into PAXgene Blood RNA tubes (QIAgen, Crawley UK). Whole-blood RNA was extracted using the PAXgene Blood RNA Kit including DNAse I treatment (QIAgen). For the test set cohort peripheral vein blood was drawn directly into Tempus™. Blood RNA tubes (Applied Biosystems Inc.). Whole-blood RNA was extracted according to manufacturer's instructions. Total RNA samples were subjected to gene expression analysis by RT-PCR and microarrays.

Samples for mRNA Studies:

95 samples from the training set were used that consisted of: 13 samples from 10 Tol-DF patients, 16 samples from 11 s-LP patients, 8 samples from 8 s-nCNI patients, 40 samples from 28 s-CNI patients, 10 samples from 9 CR patients and 8 samples from 8 HC. As the test set 142 samples were used that consisted of: 31 samples from 23 Tol-DF patients, 14 samples from 11 Mono patients, 52 samples front 34 s-CNI patients, 25 samples from 18 CAN patients and 20 samples from 20 HC.

### RNA Quality Control:

Quality and integrity of PAXgene® (training set) and Tempus™ (test set) purified RNA were determined using the Agilent RNA 6000 Nano Kit on the Agilent 2100 Bioanalyzer (Agilent Technologies). RNA was quantified by measuring A260 nm on the ND-1000 Spectrophotometer (NanoDrop Technologies).

### RNA Amplification and Labelling:

Sample labeling was performed as detailed in the “One-Colour Microarray-Based Gene Expression Analysis” protocol (version 5.5, part number G4140-90040). Briefly, 0.5 μg of total RNA was used for the amplification and labeling step using the Agilent Low RNA Input Linear Amp Kit (Agilent Technologies) in the presence of cyanine 3-CTP. Yields of cRNA and the dye incorporation rate were measured with the ND-1000 Spectrophotometer.

### Hybridization of RISET 2.0 Agilent Custom Microarrays:

All whole blood samples were hybridized on the RISET 2.0 microarray platform. This is a custom Agilent 8×15K 60 mer oligonucleotide microarray comprising 5,069 probes represented in triplicates. Probes selected corresponded to 4607 genes with a valid Entrez Gene ID and an additional 407 probes which could not be assigned to a valid Entrez Gene ID. Probe design was optimised for the detection of multiple transcript variants of a gene, on optimized hybridization properties of the probes, and avoiding crosshybridization. The hybridization procedure was performed after control of RNA quality and integrity and according to the “One-Colour Microarray-Based Gene Expression Analysis” protocol using the Agilent Gene Expression Hybridization Kit (Agilent Technologies). Briefly, 0.6 μg Cy3-labeled fragmented cRNA in hybridization buffer was hybridized overnight (17 hours, 65° C.) to RISET 2.0 microarrays. Following hybridization, the microarrays were washed once with Agilent Gene Expression Wash Buffer 1 for 1 min at room temperature followed by a second wash with preheated (37° C.) Agilent Gene Expression Wash Buffer 2 containing 0.005% N-lauroylsarcosine for 1 min. The last washing step was performed with acetonitrile for 30 sec.

### Scanning and Data Analysis:

Fluorescence signals of the Agilent Microarrays were detected using Agilent's Microarray Scanner System (Agilent Technologies, Inc.). The Agilent Feature Extraction Software (FES v.9.5.1.1) was used to read out and process the microarray image files. To determine differential gene expression, FES derived output data files were further analyzed using the Rosetta Resolver gene expression data analysis system (v.7.1.0.2., Rosetta Inpharmatics LLC). First, an artificial common reference was computed from all samples included in the IOT dataset. Using this baseline, log 2 ratios were calculated for each gene and sample. Additionally, p-values indicating the reliability of an observed difference between a sample and the common reference were calculated for each gene applying the universal error model implemented in the Rosetta Resolver software (34).

### Annotation Enrichment Analysis:

Lists of genes found to be discriminatory between different sample groups, and common to both study sets, were analysed for a statistically significant enrichment of biological pathway annotation terms in comparison to the complete RISET 2.0 microarray configuration. Term enrichment relative to the expected background distribution was scored using Fisher's exact test. Annotations were derived from different sources, e.g. Gene Ontology (GO, www.geneontology.org), signaling pathway membership, sequence motifs, chromosomal proximity, literature keywords, and cell-specific marker genes.

### Quantitative RT-PCR Analysis:

200 ng of whole blood total RNA was reverse transcribed using the qPCR 1st Strand synthesis kit (Stratagene) and synthesised cDNA was subjected to RT-PCR analysis.

### Microarray Data Validation:

A selected set of genes identified by microarray gene expression analysis were validated by quantitative RT-PCR. Quantitative RT-PCR was performed for the following genes using pre-made TaqMan panels from Applied Biosystems. Hs01017452_{—}1 B lymphoid tyrosine kinase (BLK), Hs00236881_{—}1CD79b molecule (CD79b), Hs01099196_{—}1 heparan sulfate (glucosamine) 3-O29 sulfotransferase 1 (HS3ST1), Hs01592483_{—}1 SH2 domain containing 1B (SH2D1B) Hs00172040_{—}1 Tcell leukaemia (TCL1A).

### Other Assays Screened in the Training Set:

The inventors also performed indirect pathway IFNγELISpot, and direct and indirect pathway tram-vivo DTH assays. RT-PCR amplification for cytokine genes was performed on direct and indirect pathway cultures of donor and recipient cells and TCR-repertoire profiling was achieved by TCR-Landscape analysis (data not shown).

### Statistics:

Non-parametric tests were used to estimate statistical significance as n<20 in many group comparisons and data did not conform to a normal distribution. Wilcoxon signed rank test was used to compare responses within the same group of patients. Mann-Whitney U tests were used to compare medians between patients groups. To compare associations between clinical variables, usually recorded as categorical data and presence or absence of anti-HLA antibodies, we used the Fisher Exact test. Two sided p values were used to indicate a significant difference when it was <0.05.

### Statistical Analysis of Microarrays and Biomarkers:

Significantly altered expression detected by microarray was statistically determined using four-class analysis and the Kruskal-Wallis test with Benjamini-Hochberg adjustment for False Discovery Rate (FDR) at 1%. The inventors chose a non-parametric test for this analysis as the data in some cases appeared to deviate from normality. A similar procedure was used to rank the biomarkers (tested on the log-scale, with missing values set equal to the sample-wide mean). To evaluate the predictive power of a number of variables to detect tolerant patients the inventors used receiver operating characteristic (ROC) curves. To build these, firstly four class analysis identified differentially expressed probes of Tol-DF within the training set and were ranked using the Kruskal-Wallis test. Then the top-most significantly differentially expressed probes were added in a binary regression model, and used to perform classification within sample. The binary regression procedure was used to compute probabilities p[1], . . . , p[n] of being a Tol-DF patient for each subject. The ROC curve was produced by varying a probability threshold between zero and one; for each value of the threshold t, a 2×2 classification table of Actual class versus Predicted class for subject i set equal to “Tol-DF” if p[i]>t. Bootstrap resampling of the subjects indicated that the within-sample classification, results were robust. For the test set, the same probes from the training set analysis were used.

### Tolerant Renal Transplant Patient Demographics:

The training set comprised of 71 European kidney transplant recipients and 19 age and sex-matched healthy controls (Table 1). The Tol-DF group had a high percentage of cadaveric donors (7 out of 11), a high degree of HLA mismatching (median mismatches 4.0), were predominantly male (9 out of 11), had varied causes of end stage renal failure and some evidence of sensitising events, such as blood transfusions and previous transplants (Table 2). These patients had relatively uneventful posttransplant courses with only 1 patient having a documented episode of acute cellular rejection (ACR). The period of being immunosuppression-free varied from 1 to 21 years. The Tol-DF group of the test set (Table 3) consisted of 24 patients most of whom had received their transplant from a highly HLA-matched living donor (median mismatches 0.0) and had ceased to take immunosuppression for periods from 1 to 32 years.

Tol-DF recipients displayed increased numbers of B and NK lymphocytes. As shown in FIG. 1, Tol-DF patients of the training set displayed an increased percentage of peripheral blood B and NK cells, and a corresponding decrease in the percentage of T cells. When expressing the percentages of B cells and T cells as a ratio, Tol-DF patients displayed the highest ratio compared to all other study groups including HC. For 6 Tol-DF patients and 10 s-CNI patients it was also possible to calculate the absolute number of cells per lymphocyte subset. This showed that the altered ratio was due to an expansion in B and NK cell numbers and not a reduction in T cell numbers, as none of the Tol-DF group were lymphopenic (Table 2). In line with the findings in the training set, Tol-DF patients of the test set also showed elevated percentages of peripheral blood B cells and a higher ratio of B:T cell percentages (FIGS. 1E & H) compared to all other groups except HC. Given the distinct increase in peripheral blood B cells detected in Tol-DF patients, B cell subset analysis (FIG. 8) and cytokine production (FIG. 9) were further assessed in selected patients from both study sets. The Tol-DF group displayed a trend in redistribution of B cell subsets, with a decreased late-memory pool and concomitant increase in transitional and naive B cell subsets. When examining the percentages of B cell subsets as a ratio, Tol DF patients were found to have a significantly lower proportion of memory and higher proportion of transitional B cells compared to CR patients. A significant proportion of B cells from Tol-DF patients were found to produce TGFβ upon in vitro stimulation, rather than IL-10 or IFNγ. However, no significant differences in production of IL-10 were detected for any study group. The capacity of B cells from each patient group to produce either cytokine on stimulation was analysed by calculating a ratio of the number of B cells producing each cytokine. This suggested that B cells of Tol-DF patients had a skewed cytokine response, with a higher propensity for TGFβ production than B cells from other study groups.

Tolerant recipients had fewer activated CD4+T cells in peripheral blood. Expression of CD25 by CD4+ T cells was analysed as described above. Tol-DF patients in the training set were found to have significantly lower percentages of circulating CD4+CD25int T cells, broadly thought of as activated T cells (9, 10) (FIG. 2A), compared to HC, s-LP, s-nCNI and CR groups. Interestingly, significant differences in the percentages of CD4+CD25hi regulatory T cells were only detected between patients on full immunosuppression with s-CNI and other a (FIG. 2B). Similar results were also found in the test set, with Tol-DF patients having significantly lower percentages of CD4+CD25int T cells compared to s-CNI and CAN groups, but again no differences in the percentages of CD4+CD25hi Tregs wore detected between Tol-DF and any other study group (FIGS. 2C and 2D). Statistical comparisons between other groups are shown in Table 5. When we tested the ability of enriched CD4+CD25hi T cells to suppress autologous T cell proliferation induced by polyclonal stimulation, no significant differences were found between any of the patient groups or healthy controls (data not shown). Furthermore, Tol-DF patients did not display higher percentages of other regulatory T cell subsets such as CD3+CD8+CD28− or CD3+CD4−CD8− T cells (data not shown).

The majority of tolerant recipients did not have detectable anti-donor HLA specific antibodies. Serum non-donor specific antibodies (NDSA) were detectable in some patients from all study groups of the training set (FIG. 3A) by Luminex xMAP analysis. Within this cohort, no Tol-DF patients had detectable donor-specific antibodies (DSA), whereas all other groups had some patients with detectable DSA, with almost half of the CR patients having detectable both donor, and non-donor specific anti-HLA class I and class II antibodies. Similar to the training set, only 1 of 22 Tol-DF patients within the test set had detectable DSA (data not shown). Interestingly, in general DSA-positive patients had worse graft function than DSA-negative patients, with an estimated glomerular filtration rate of 31 (range 17-87) in DSA-positive patients compared to 60 (range 13-94) in DSA-negative patients. The possible pathogenicity of detected anti-donor antibodies was tested in the training set (FIG. 3B). In 7 of 20 patients with anti-class I antibodies and 4 of 13 patients with anti-class II antibodies, the inventors found complement-fixing isotypes (IgG1 and IgG3); the remaining positive cases were exclusively of non-complement-fixing isotypes. Detection of NDSA anti-class I and anti-class II antibodies was significantly associated with having received a previous transplant and having detectable panel reactive antibodies before transplant (Fisher Exact test p<0.05), but not with previous pregnancies, blood transfusions, graft dysfunction or episodes of ACR. In contrast, DSA anti-class II antibodies were associated with previous episodes of ACR and the number of HLA mismatches between donor and recipient (Fisher Exact test p<0.05).

Tolerant patients have lower frequencies of direct pathway anti-donor IFNγCD4+ T cell responses. Comparison of direct pathway CD4+ T cell anti-donor and anti-3^{rd} party (equally mismatched to donor) responses was assessed by IFNγ ELISpot. Tol-DF patients had significantly higher ratios of responder anti-donor:anti 3^{rd}-party frequencies indicating donor-specific hyporesponsiveness, compared to all other stable patient groups within the training set (FIG. 4A; individual responder frequencies against donor and 3^{rd} party are shown in FIG. 10). Donor-specific hyporesponsiveness was not mediated by Tregs, as depletion of CD25+ cells from responder T cells did not result in an increase in responder frequencies (data not shown). As the Tol-DF group of the test set was frequently completely HLA matched with their donor, anti-donor and anti-3^{rd} party IFNγresponses were generally very low (responder frequencies>1/200,000). Despite this, the trend in anti-donor responses in this Tol-DF group was generally reproduced although a significant difference compared to other groups was not detected (FIG. 4B).

Tolerant recipients displayed a higher ratio of expression of FoxP3 and α-1,2-mannosidase genes in peripheral blood Whole blood gene expression levels of FoxP3 and α-1,2-mannosidase, both of which have been shown to correlate with anti-donor immune reactivity after transplantation (11) were analysed by qRT-PCR (FIG. 11). When calculating the ratio of FoxP3 and α-1,2-mannosidase expression, a significant difference was detected between Tol-DF and the CR and HC groups of the training set (FIG. 5A). The patient groups displaying the highest ratio were HC, s-LP and Tol-DF whereas the ratio was dramatically lower in CR (Mann-Whitney U test p values for comparisons between groups other than Tol-DF are shown in Table 6). This ratio significantly correlated with eGFR and inversely correlated with serum creatinine (Pearson Coefficient: 0.372 p=0.002 and −0.299 p=0.014 respectively, data not shown). When the same analysis was performed on the test set, the ratio in Tol-DF patients was significantly higher than in all other patient groups except HC (FIG. 5B). Combining the training and test set observations shows that tolerance is associated with a high ratio of peripheral blood FoxP3 and α-1,2-mannosidase expression.

Tolerant patients exhibited a distinct gene expression profile. The RISET 2.0 custom microarray, designed with a focus on transplantation research, was assembled by the inclusion of 5,069 probes and used to analyse the expression of 4607 genes (valid Entrez Gene ID) in peripheral blood samples. A four-class analysis of microarray data was performed on the training set (FIG. 6). Significantly altered gene expression detected between Tol-DF patients and other comparator groups, stable recipients (s-CNI, s-nCNI and s-LP), CR and HC, was statistically determined using the Kruskal-Wallis non-parametric test with adjustment for False Discovery Rate (FDR) at 1% (12). The HC group was included in this analysis in order to address the lack of immune-suppression in Tol-DF patients compared to the other study groups. Two hundred and sixty probes, corresponding to 255 genes, were identified as being significantly differentially expressed between the study groups. When a similar analysis was performed on the test set, 1,378 probes, corresponding to 1352 genes, with significantly altered expression were identified, with 174 probes (170 genes) found to be common between both the training and test sets (Table 7).

Microarray expression was validated by qRT-PCR analysis of several probes that were highly ranked within the list, and including probes detected to be either down- or up-regulated. Expression of all the genes was highly correlated using both assays (FIGS. 12 A-E) and qRT-PCR quantitated expression of the selected genes was significantly different to at least one of the other patient groups, depending on the gene studied (FIG. 12 F-J). Interestingly, the median expression levels in Tol-DF patient samples for all selected genes was very similar between the training and test set, although due to the higher sample number in the test set their correlation coefficients were generally higher (FIG. 13 A-E). Furthermore, gene expression in Tol-DF patients was significantly different to most of the other groups for 4 of 5 genes tested (FIGS. 12 & 13 F-J). Median probe expression values for top ranked probes are shown in Table 8.

Gene expression diagnostic capabilities for a more precise quantitative approach to gene expression analysis, with the utility to identify tolerant from non-tolerant individuals, were investigated by the inventors using the top ranked genes identified by microarray analysis, excluding any overlapping probes for any single gene (e.g. TCL1A ranked 2 and 4, excluding probe ranked 4), in an additive binary regression model to build ROC curves. These probes were used to build a gene expression signature to specifically identify Tol-DF patients by firstly producing predicted classes (within-sample) and hence a classification for each individual. For this analysis, two-class ROC curves (tolerant vs non-tolerant) were built by both including and excluding the HC group from the non-tolerant comparator groups. This was done because whilst the comparison of healthy controls to tolerant individuals is of interest in identifying tolerance-specific gene expression, in the context of developing a clinical diagnostic test for tolerance in renal transplant patients, this comparison is not useful. The corresponding ROC curve built excluding HC (FIG. 6A) and based on the expression of the top 10 ranked genes (Table 4) delivered a peak specificity and sensitivity of 1, with a threshold of 0.01, a corresponding positive predictive value (PPV) and negative predicative values (NPV) of 100% within the training set (ROC including HC; threshold 0.2, specificity 0.853, sensitivity 0.923). Although 3 genes and 6 genes were sufficient to deliver good discrimination of tolerant patients within the training set, the top 10 ranked genes were selected for use, as they improved the specificity and sensitivity of subsequent ROC analysis of the test set (FIG. 6B). Within sample analysis of the test set, delivered a specificity of 0.890 and sensitivity of 0.806, with a threshold of 0.35, a PPV and NPV of 71% and 93%, respectively (ROC including HC; threshold 0.23, specificity 0.801, sensitivity 0.806).

The inventors performed annotation enrichment analyses on the set of 174 overlapping probes identified between the training and test sets. The majority of genes found to have any significant association with annotated pathways were enriched within B cell related pathways (Table 9). In line with these data, of the top 11 ranked probes, corresponding to 10 genes, 6 genes are described to be expressed by B cells or related to B cell function (Table 4). In addition to the B cell related pathways enriched within this probe list, other pathways were also significant, including protein-tyrosine kinases, generation of secondary signaling messenger molecules and other T cell activation related pathways (Table 9).

### Cross-Platform Biomarker Diagnostic Capabilities.

All assays described in the Materials and Methods section were tested in parallel for their diagnostic ability to distinguish Tol-DF patients from all other study groups. Assays performed on the test set were those that were highly predictive of tolerance within the training set and are discussed above. By combining the various biomarkers which indicate the presence of tolerance, the inventors expected that it was possible to significantly improve the diagnostic ability of any such individual test. This was indeed observed for the test set. Indeed when biomarkers and microarray data were analysed in combination, using 1) the ratio of B/T lymphocyte subsets, 2) the percentage of CD4+CD25int T cells, 3) the ratio of anti-donor to anti-3^{rd} party ELISpot frequencies, 4) the ratio of FoxP3/α-1,2-mannosidase expression and 5) a signature of the top 10 ranked genes, the specificity and sensitivity for the training set was 1, with a threshold of 0.01, which implied PPV and NPV of 100% (FIG. 7A). When analysing the test set a peak specificity of 0.923 and a sensitivity of 0.903 were obtained with a threshold of 0.27, PPV of 80% and NPV of 96% (FIG. 7B), which improved the diagnostic capacity compared to that obtained with gene expression alone. Therefore, application of a cross-platform biomarker signature improves the ability to identify bona fide tolerance, as in addition to gene expression and phenotype analysis, it can also take into consideration an individual's immunological functional state, which may be more closely related to describing the mechanistic basis of tolerance. In this respect, studying patient T cell and B cell responses are useful approaches and may also be used as biomarkers in the present invention. The utility of this cross-platform biomarker signature lies in its ability to identify renal transplant patients who may be unknowingly operationally tolerant. As shown in FIGS. 7 C and D, 5 stable recipients of the test set could be identified to have the tolerant signature, and therefore may benefit from managed immunosuppression weaning. Interestingly, 2 CAN patients of the test set were also identified as haying a high probability of being tolerant. This finding may be explained by differences in the clinical assessment, of chronic rejection, as unlike the CR group of the training set, CAN patients were not proven by biopsy to have immune-mediated rejection, but were defined on the basis of poor graft function. It is possible that the cross-platform biomarkers used to test these patients have sufficient sensitivity to detect subtle differences between these two patients groups, a property which may be revealed by serial immune monitoring of patients such as these over time,

### Statistical Data

The statistician calculated the following sensitivities and specificities using the training set:

| | Threshold | Specificity | Sensitivity |
|---|---|---|---|
| CD4.CD25 (FLOW CYTOMETRY) | 0.14 | 0.695122 | 0.615385 |
| B.T (FLOW CYTOMETRY) | 0.12 | 0.804878 | 0.692308 |
| FoxP3:1,2αMannosidase (RT-PCR) | 0.18 | 0.841463 | 0.461538 |
| Donor Specific CD4+ (IFNgamma | 0.13 | 0.768293 | 0.538462 |
| ELISpot) | | | |

The same calculations were then made using the test set:

| | Threshold | Specificity | Sensitivity |
|---|---|---|---|
| CD4.CD25 (FLOW CYTOMETRY) | 0.23 | 0.846847 | 0.419355 |
| B.T (FLOW CYTOMETRY) | 0.23 | 0.837838 | 0.548387 |
| FoxP3:1,2αMannosidase (RT-PCR) | 0.17 | 0.738739 | 0.677419 |
| Donor Specific CD4+ (IFNgamma | 0.21 | 0.153153 | 0.903226 |
| ELISpot) | | | |

The statistician calculated the following sensitivities and specificities for the listed genes using the training set:

| | Thresh | Spec | Sens |
|---|---|---|---|
| No gene | 0 | 0 | 1 |
| CD79B | 0.16 | 0.837838 | 0.923077 |
| TCL1A | 0.14 | 0.783784 | 0.846154 |
| HS3ST1 | 0.15 | 0.810811 | 0.769231 |
| SH2D1B | 0.21 | 0.851351 | 0.923077 |
| MS4A1 | 0.13 | 0.77027 | 0.846154 |
| TLR5 | 0.12 | 0.689189 | 0.923077 |
| THC2438936 | 0.15 | 0.797297 | 0.846154 |
| PNOC | 0.12 | 0.72973 | 0.846154 |
| SLC8A1 | 0.18 | 0.743243 | 0.692308 |
| FCRL2 | 0.11 | 0.716216 | 0.846154 |

The same calculations were then made using the test set:

| | Thresh | Spec | Sens |
|---|---|---|---|
| No gene | 0 | 0 | 1 |
| CD79B | 0.33 | 0.868132 | 0.806452 |
| TCL1A | 0.24 | 0.813187 | 0.870968 |
| HS3ST1 | 0.28 | 0.802198 | 0.870968 |
| SH2D1B | 0.26 | 0.681319 | 0.741935 |
| MS4A1 | 0.26 | 0.714286 | 0.806452 |
| TLR5 | 0.27 | 0.725275 | 0.741935 |
| THC2438936 | 0.3 | 0.78022 | 0.806452 |
| PNOC | 0.28 | 0.758242 | 0.774194 |
| SLC8A1 | 0.23 | 0.571429 | 0.774194 |
| FCRL2 | 0.29 | 0.769231 | 0.774194 |

In order to select the best subset of genes and additional biomarkers that would provide the best predictive value, as well as good generalizability, additional analysis was carried out. First, the best subset of each size (1 to 14 biomarkers) was selected based on the Akaike's Information Criterion. The biomarkers selected for each subset are shown in the table below.

| | V1 | V2 | V3 | V4 | V5 |
|---|---|---|---|---|---|
| 1 | SH2D1B | NA | NA | NA | NA |
| 2 | SH2D1B | PNOC | NA | NA | NA |
| 3 | SH2D1B | TLR5 | PNOC | NA | NA |
| 4 | CD4.CD25 | SH2D1B | TLR5 | PNOC | NA |
| 5 | CD4.CD25 | Donor Specific | SH2D1B | TLR5 | PNOC |
| | | CD4+ | | | |
| 6 | CD4.CD25 | TCL1A | SH2D1B | MS4A1 | SLCBA1 |
| 7 | CD4.CD25 | HS3ST1 | SH2D1B | MS4A1 | TLR5 |
| 8 | CD4.CD25 | HS3ST1 | SH2D1B | MS4A1 | TLR5 |
| 9 | CD4.CD25 | TCL1A | HS3ST1 | SH2D1B | MS4A1 |
| 10 | CD4.CD25 | B.T | TCL1A | HS3ST1 | SH2D1B |
| 11 | CD4.CD25 | B.T | CD79B | TCL1A | HS3ST1 |
| 12 | CD4.CD25 | B.T | CD79B | TCL1A | HS3ST1 |
| 13 | CD4.CD25 | B.T | Donor Specific CD4+ | CD79B | TCL1A |
| 14 | CD4.CD25 | B.T | FoxP3:1,2αMannosidase | Donor Specific | CD79B |
| | | | | CD4+ | |

| | V6 | V7 | V8 | V9 | V10 | V11 | V12 | V13 | V14 |
|---|---|---|---|---|---|---|---|---|---|
| 1 | NA | NA | NA | NA | NA | NA | NA | NA | NA |
| 2 | NA | NA | NA | NA | NA | NA | NA | NA | NA |
| 3 | NA | NA | NA | NA | NA | NA | NA | NA | NA |
| 4 | NA | NA | NA | NA | NA | NA | NA | NA | NA |
| 5 | NA | NA | NA | NA | NA | NA | NA | NA | NA |
| 6 | FCRL2 | NA | NA | NA | NA | NA | NA | NA | NA |
| 7 | SLC8A1 | FCRL2 | NA | NA | NA | NA | NA | NA | NA |
| 8 | THC2438936 | SLC8A1 | FCRL2 | NA | NA | NA | NA | NA | NA |
| 9 | TLR5 | THC2438936 | SLC8A1 | FCRL2 | NA | NA | NA | NA | NA |
| 10 | MS4A1 | TLR5 | THC2438936 | SLC8A1 | FCRL2 | NA | NA | NA | NA |
| 11 | SH2D1B | MS4A1 | TLR5 | THC2438936 | SLC8A1 | FCRL2 | NA | NA | NA |
| 12 | SH2D1B | MS4A1 | TLR5 | THC2438936 | PNOC | SLC8A1 | FCRL2 | NA | NA |
| 13 | HS3ST1 | SH2D1B | MS4A1 | TLR5 | THC2438936 | PNOC | SLC8A1 | FCRL2 | NA |
| 14 | TCL1A | HS3ST1 | SH2D1B | MS4A1 | TLR5 | THC2438936 | PNOC | SLC8A1 | FCRL2 |

A binary regression model was estimated for each of those subsets, and cross validation was used to establish the stability of the solution, in order to avoid overfit in the test set. FIG. 14 shows the results of the cross-validation. The results of the cross validation suggest that the optimal solution should include a small number of markers (for example 2 to 5 or preferably 2 to 3), since the models seem to start overfilling to the specific characteristics of the test set with the inclusion of additional markers.

To confirm this, the binary regression models including the best subsets of each size were used to estimate ROC curves, and the corresponding optimal sensitivity and specificity in the training set.

### Training Set

| | Threshold | Specificity | Sensitivity |
|---|---|---|---|
| 1 | 0.21 | 0.851351 | 0.923077 |
| 2 | 0.12 | 0.932432 | 1 |
| 3 | 0.11 | 0.905405 | 1 |
| 4 | 0.11 | 0.932432 | 1 |
| 5 | 0.34 | 0.986486 | 1 |
| 6 | 0.01 | 1 | 1 |
| 7 | 0.01 | 1 | 1 |
| 8 | 0.01 | 1 | 1 |
| 9 | 0.01 | 1 | 1 |
| 10 | 0.01 | 1 | 1 |
| 11 | 0.01 | 1 | 1 |
| 12 | 0.01 | 1 | 1 |
| 13 | 0.01 | 1 | 1 |
| 14 | 0.01 | 1 | 1 |

Subsequently, the probability of tolerance was estimated for the patients in the test set, by using the coefficients obtained in the training set for each subset size. These probabilities where used in combination with the optimal cutoff (also estimated in the training set) to compute the sensitivity and specificity in the test set.

| | Thresh | Spec | Sens |
|---|---|---|---|
| 1 | 0.21 | 0.758242 | 0.612903 |
| 2 | 0.12 | 0.868132 | 0.677419 |
| 3 | 0.11 | 0.879121 | 0.709677 |
| 4 | 0.11 | 0.923077 | 0.612903 |
| 5 | 0.34 | 0.967033 | 0.387097 |
| 6 | 0.01 | NA | NA |
| 7 | 0.01 | 0.967033 | 0.129032 |
| 8 | 0.01 | 0.967033 | 0.193548 |
| 9 | 0.01 | 0.923077 | 0.645161 |
| 10 | 0.01 | 0.967033 | 0.225806 |
| 11 | 0.01 | 0.846154 | 0.193548 |
| 12 | 0.01 | 0.967033 | 0.193548 |
| 13 | 0.01 | 0.967033 | 0.193548 |
| 14 | 0.01 | 0.967033 | 0.225806 |

These results confirm those of the cross-validation, and support the use of a model with preferably 2, 3, 4 or 5 biomarkers more preferably 2 or 3 biomarkers, to best predict the probability of tolerance in individual patients.

### Discussion

The inventors have developed a set of biomarkers that distinguish tolerant it transplant recipients from patients with stable renal function under different degrees of immunosuppression, patients undergoing chronic rejection and healthy controls. Biomarkers identified in a training set of tolerant patients bate been validated in an independent test set. The inventors have found an expansion of B and NK cells in peripheral blood of drug-free tolerant recipients, which is similar to the findings of a previous study on a smaller cohort of similar patients (13). Microarray analysis also revealed a clear and strong B cell bias of genes with altered expression between Tol-DF and the other groups. In particular, it has been found that the combination of the SH2D1B, TLR5 and PNOC genes provides a very effective test for determining an individual's tolerance. The role of T cells in initiating and maintaining allograft rejection (14, 15) and tolerance (16) has been clearly established, whereas the role of B cells and the mechanisms whereby they may contribute to the tolerant state have yet to be elucidated. Interestingly, a murine study of transplantation tolerance, induced by anti-CD45RB therapy has shown a mechanistic role for B cells (17). Recent data have also shown the ability of naive B cells, following antigen-specific cognate interactions, to induce regulatory T cells that inhibit graft rejection in a murine model of heart transplantation (18). Whilst no significant increase in Br-1 (IL-10 producing B cells) was detected in any patient group within this study, data presented here show altered ratios of B cell transitional and late-memory populations, relative increase of TGFβ producing B cells, absence of serum donor-specific antibodies and donor-specific direct T cell hyporesponsiveness in tolerant recipients. These observations allow speculation that renal transplant tolerance may be associated with alterations in both T-cell and B-cell mediated functions. A recent study by Porcheray et al., studying both B cell and T cell immunity in combined kidney and bone marrow transplant recipients, has however demonstrated the uncoupling of T cell and B cell anti-donor immunity in some of their studied tolerant patients (19). In this respect, the B cell signature observed in tolerant renal patients in this study may indicate an important role for B cells in promoting tolerance.

Monitoring of anti-donor responses using functional assays has demonstrated that hyporesponsiveness of direct pathway T cells develops over time after solid organ transplantation (20, 21). In the clinical context, enumerating the frequency of anti-donor T cells has proven useful in steroid withdrawal protocols (22). In the present study, measuring anti-donor direct pathway responses by ELISpot has also proven useful, where determining the ratio of responses against donor and third party T cells reveals donor specific hyporesponsiveness in tolerant patients. This test, however, is more useful when donor and recipient have several HLA-mismatches. Similar studies to this have focused on gene profiling of tolerant liver (23, 24) and also tolerant kidney recipients (25, 26). The set of genes that were differentially expressed in those studies differ to those identified herein and are not as effective in determining whether an individual is immunologically tolerant. This possibly reflects differences in the organ, the patient groups, the RNA source and preparation protocol, or the analysis platform used. Indeed the microarray used in this study was selectively designed based on both published and unpublished data to have a transplantation focus, and therefore included a significant number of immune response related probes.

The two of the most highly ranked genes associated with tolerance found within the training set, TCL1A (rank 2) MS4A1 (CD20) (rank 5), are both B cell related genes, MS4A1 has previously been identified by Brand et al., (25) to be associated with tolerant renal transplant patients.

A possible interpretation of the tolerant signature described by this study could be that the immunological biomarkers detected are merely due to the lack of drug-mediated immune suppression in the Tol-DF group. To address this possibility, the study groups of the training set were specifically selected to include stable renal transplant patients on distinct immunosuppressive regimes and healthy controls as immune suppression-free subjects. Although clear differences between the healthy control and Tol-DF groups were observed in the training set, these differences were not reproduced in the test set, a finding which may be attributed to the fact the mechanisms of tolerance may be more subtle within the test set, where tolerant recipients are highly HLA-matched to their donors, in contrast to the training set. As all of these study groups have been taken into consideration, the combination of biomarkers described here appears to be a specific description of transplant tolerance, rather than simply a consequence of the absence of immunosuppression. It is pertinent to observe that whilst detailed comparison of tolerant patients and healthy controls may reveal more about the mechanistic basis of tolerance, in the clinical context, this comparison is not relevant.

An interesting comparison is that of Tol-DF and s-LP patient groups of the training set, which differ in the use of 10 mg/day of prednisone, considered by many clinicians as quasi-physiological. The s-LP group had a higher proportion of female recipients, a higher percentage of cadaveric donors, and poorer kidney function than the Tol-DF group. Rather counter-intuitively, in most of the assays described there are clear differences between these two groups in immunophenotype, anti-donor responses, FoxP3/α-1,2-mannosidase ratio and gene expression. This supports the notion that steroid monotherapy can induce a significant difference in the patient's immune status that can be evidenced by biomarkers. One of the Tol-DF patients within the training set had received a bone marrow donation 4 years prior to kidney transplantation from the same donor. Immune suppression was initially withdrawn from this patient as evidence of chimerism was detected. As the mechanisms of tolerance induction could be different in this patient, biomarker and ROC curve analysis was performed by inclusion and exclusion of this patient, however this patient did not appear as an outlier within the tolerant group in any of the assays studied.

The utility of this tolerant signature depends on its ability to identify transplant recipients that can safely be weaned from immunosuppression. The inventors have now developed a specific and sensitive set of biomarkers, which when combined, can identify tolerant renal allograft recipients and also several renal transplant recipients on immunosuppressive drugs. Validation of these biomarkers has been achieved using a completely independent set of patients, and this validation is reinforced the fact that the test set was derived from a genetically different population, and that there were also differences in the collection and processing of test set and training set samples. The biomarkers can be implemented as a decisional tool in the clinical setting, which may allow tailored and safe clinical posttransplantation management of renal allograft recipients.

### Further Validation Using RT-PCR

In order to further validate the present method of determining tolerance the inventors performed a further study (the “GAMBIT” study) on a different patient group.

### New Study Groups:

Tolerants: new patients that have been completely off immunosuppression for longer than one year with <10% CRT rise since baseline before weaning. (Corresponds to Index group of the IOT study)

Stable: Adult kidney transplant recipients, with stable function, that have been transplanted for longer than 5 years, that are maintained on any immunosuppression therapy and that have had overall stable kidney function (<15% change in mean eGFR) in the last 5 years. (Corresponds to control groups 1, 2 and 3 of the IOT study)

Chronic Rejection: Adult and paediatric kidney transplant recipients, more than 1 year posttransplant with increasing dysfunction that have undergone a graft biopsy in the previous 3 months and have been classified as having immunologically-driven chronic allograft nephropathy. (Corresponds to control group 4 of the IOT study).

in this study the inventors collected new samples from the following patient groups:

33 samples from Stable patients
12 samples from patients on Chronic Rejection, and
5 samples from Tolerant patients
1 sample from a Healthy Control
1 sample from a patient who has lost tolerance

RT-PCR was performed on the 10 genes selected using the following protocol.

### RT-PCR Protocol

Whole blood was collected directly from the peripheral vein into “Tempus Tubes™” (ABI cat number: 4342792), containing a solution that lyses cells and stabilizes mRNA. The tubes were stored at −20° C. until use.

Whole blood RNA was extracted using the Tempus Spin RNA isolation Kit (ABI cat number: 4380204). The quantity and quality of the mRNA was measured using the ND 1000 Spectrophotometer (NanoDrop Technologies). The RNA was then stored at −80° C.

1 μg of whole blood total RNA was reverse transcribed using the ABI Taqman Reverse Transcription synthesis kit (ABI cat number: 4304134) into cDNA for immediate use. cDNA was subjected to RT-PCR analysis using the primers and probes, shown below, in 384-well plates (ABI cat number: 4306737) in 20 μl reaction volumes per well.

| | | Non-inventoried Sequences | |
|---|---|---|---|
| Gene name | ABI Hs # | (source: IOT) | |
| HPRT | N/A | fw: | 5′-agtctggcttatatccaacacttcg-3′ |
| | | rev: | 5′-gactttgctttccttggtcagg-3′ |
| | | pr: | 5′-tttcaccagcaagcttgcgaccttga-3′ |

| | | |
|---|---|---|
| SH2D1B | Hs01592483_m1 | N/A |
| TLR5 | Hs00152825_m1 | N/A |
| PNOC | Hs00173823_m1 | N/A |
| CD79B | Hs00236881_m1 | N/A |
| FCRL1 | Hs00364705_m1 | N/A |
| FCRL2 | Hs00229156_m1 | N/A |
| SLC8A1 | Hs00253432_m1 | N/A |
| TCL1A | Hs00172040_m1 | N/A |
| MS4A1 | Hs00544818_m1 | N/A |
| HS3ST1 | Hs01099196_m1 | N/A |

After the initial RT-PCR step to check the levels of expression of the genes in samples from healthy controls, and to demonstrate that the expression of the same genes is also detected in whole blood samples from patients, RT-PCR was carried out on patient cDNA for this study.

Data preprocessing steps:
Read in and merge the data from the different plates
Check for batch effects due to a change in type of plate (between 96 well plates and 384 well plates). No batch effects found.
Check wells with non-template controls to detect possible contamination in the plates.
Code CT values above 35 as undetermined.
Check the coefficient of variation across technical duplicates, considering alarming any above 3%. I have found great quality of duplicates, and no reason for concern.
Aggregate duplicates using the mean.
Calculate dCT as the difference in CT values between the gene of interest and HPRT (the control gene).
Resettle dCT to obtain only positive values using 2^{-dCT}.
Eliminate data from the gene SLC8A1 due to excessive levels of undetermined expression. This gene is very lowly expressed and badly detected by RT-PCR. It is likely that this gene was a false positive, originally selected due to outlier values.
Eliminate data from patients with missing values (only stable patients have been eliminated).

The data is produced in the form of heatmaps (not shown), wherein dendrograms show the results of unsupervised hierarchical clustering of patients using either 10 or 3 genes. It is apparent that using 10 genes does not help to group tolerant patients together, whereas using the three genes selected via cross-validation the 5 tolerant patients tend to cluster together on the right side, under the last branch of the dendrogram. Data not shown.

Box plots showing the expression levels of the 3 genes PNOC, SH2DB1 and TLR5 are shown. See FIG. 15.

There are several possible ways to combine the three genes to create a classifier to differentiate tolerant from non-tolerant patients, as will be apparent to those skilled in the art. The inventors present here the results of two classifiers: 1) a logistic regression model with main and interaction effects, and 2) a classification tree.

in order to calculate the parameters of these models the inventors used the data from stable, chronic rejectors and tolerant patients, but dichotomize the outcome as tolerant vs non-tolerant.

Results from Logistic Regression Fit:

Coefficients:

| | Estimate | Std. Error | z value | Pr(>\|z\|) |
|---|---|---|---|---|
| (Intercept) | −14.457 | 8.319 | −1.738 | 0.0822 |
| PNOC | 94.156 | 196.511 | 0.479 | 0.6318 |
| SH2DB1 | 6.289 | 4.337 | 1.450 | 0.1470 |
| TLR5 | 5.054 | 2.628 | 1.923 | 0.0545 |
| PNOC:SH2DB1 | −1.523 | 58.209 | −0.026 | 0.9791 |
| PNOC:TLR5 | −51.584 | 58.373 | −0.884 | 0.3769 |
| SH2DB1:TLR5 | −2.339 | 1.921 | −1.217 | 0.2234 |

Signif. codes: 0 ‘***’ 0.001 ‘**’ 0.01 ‘*’ 0.05 ‘.’ 0.1 ‘ ’ 1
(Dispersion parameter for binomial family taken to be 1)
Null deviance: 32.508 on 49 degrees of freedom
Residual deviance: 14.892 on 43 degrees of freedom
AIC: 28.892

The coefficients under “Estimate” column are the ones used to calculate the probability of Tolerance. See FIG. 16 for the ROC curve obtained using a logistic regression classifier, FIG. 17 shows boxplots of the probability of tolerance estimated using the logistic regression classifier.

Using this method a single chronic rejector was misclassified as tolerant, and 5 stable patients were classified as tolerant, comprising 15% of the stable population, falling within the predicted 20% who might be eligible for immunosuppression weaning.

### Regression Algorithm:

Z=−14.4574+94.156*PNOC+6.289*SH2DB1+5.054*TLR5−1.523*PNOC*SH2DB1−51.584*PNOC*TLR5−2.339*SH2DB1*TLR5

P(Tol)=e^{Z}/(e^{Z}+1)

Note: The expression of each gene is expressed as 2^{-dCT}, where dCT is calculated as the CT difference between each gene and the control gene (HPRT). A patient is classified as tolerant if P(Tol) is >0.0602.

FIG. 18 shows a classification tree estimated with the 3 genes (PNOC, SH2DB1 and TLR5). For optimal performance of the classifier, an) patient with a probability of tolerance larger than 0 is classified as tolerant. Two of the terminal nodes have associated probability of tolerance larger than 0: 1) Patients with TLR5<3.37 & SH2DB1>1.02 & TLR5<1.58 (probability of tolerance=0.600); and 2) Patients with TLR5<3.37 & SH2DB1>1.02 & TLR5>1.58 & PNOC<0.042 (probability of tolerance=0.333).

FIG. 19 shows boxplots of the classification tree's cutoffs.

FIG. 20 shows the ROC curve resulting from the use of the estimated classification tree.

The sensitivity, specificity and AUC result from classifying as tolerant any patient with a probability of tolerance larger than 0. FIG. 21 shows the number of patients of each group assigned to different probabilities.

One CR patient was misclassified as tolerant (same misclassified using regression). Equally, 5 stable patients were classified as tolerant.

These results show the success obtained using these three genes to distinguish between tolerant and non-tolerant patients. A successful performance can be achieved using different classification methods, two of which are illustrated here.

TABLE 1
Clinical and demographic characteristics of the training set (IOT cohort).

| | | | | | | | | | | | | | | Pats | Anti- |
|---|---|---|---|---|---|---|---|---|---|---|---|---|---|---|---|
| | | | | | | | | | | | Pats | Pats | Pats | on | body |
| | | | % | Post- | | | | % 1st | % | HLA- | on | on | on | Ste- | induc- |
| | N | Age ^{a} | Female ^{b} | Tx ^{c} | eGFR ^{d} | CRT ^{e} | Lymph ^{f} | Tx ^{g} | CAD ^{h} | MM ⁱ | CNI ^{j} | MMF ^{k} | Aza ^{l} | roids ^{m} | tion ⁿ |
| HC | 19 | 44 | 47 | | | | | | | | | | | | |
| | | (37-52) | | | | | | | | | | | | | |
| Tol-DF | 11 | 54 | 18 | 12 | 76.2 | 97.0 | 1.855 | 64 | 64 | 4.0 | 0 | 0 | 0 | 0 | 2 |
| | | (41-58) | | (7-23) | (58-69) | (88-119) | (1.4-2.2) | | | | | | | | |
| s-LP | 11 | 44 | 36 | 12 | 58.1 | 120.6 | 1.980 | 73 | 91 | 4.0 | 0 | 0 | 0 | 11 | 0 |
| | | (36-58) | | (9-17) | (41-70) | (107-141) | (1.5-2.3) | | | | | | | | |
| s-nCNI | 10 | 49 | 40 | 25 | 64.2 | 101.5 | 1.790 | 70 | 30 | 3.5 | 0 | 0 | 10 | 10 | 0 |
| | | (44-57) | | (23-26) | (59-74) | (87-121) | (1.6-1.9) | | | | | | | | |
| s-CNI | 30 | 42 | 47 | 6 | 56.3 | 115.0 | 1.200 | 86 | 7 | 4.0 | 22 | 11 | 14 | 29 | 5 |
| | | (37-47) | | (5-9) | (49-63) | (106-127) | (0.9-1.8) | | | | | | | | |
| CR | 9 | 52 | 33 | 5 | 19.0 | 312.0 | 1.335 | 78 | 44 | 4.0 | 6 | 4 | 3 | 7 | 5 |
| | | (31-54) | | (3-11) | (13-31) | (208-382) | (1.2-1.5) | | | | | | | | |
| All pats | 71 | 44 | 38 | 9 | 58.1 | 117.0 | 1.500 | 77 | 38 | 4.0 | 28 | 15 | 27 | 57 | 13 |
| | | (37-56) | | (5-18) | (43-70) | (98-143) | (1.2-1.9) | | | | | | | | |

^{a, c, d, e, f} Medians and interquartile range per group are shown.
^{a} Age (years);
^{b} Percentage of females in each group;
^{c} Time after transplantation (years);
^{d} estimated Glomerular Filtration Rate using MDRD function, http://nephron.org/mdrd_gfr_si;
^{e} Serum Creatinine values (normal range 60-105 μmol/L);
^{f} Peripheral blood lymphocyte counts (×10⁹ cells per L);
^{g} Percentage of patients with their first transplant;
^{h} Percentage of patients with cadaveric donors;
ⁱ Median number of HLA A, B, C, DR, and DQ mismatches between donor and recipient (maximum 10);
^{j} Number of patients on CNI at the time of sample collection;
^{k} Number of patients on mycophenolate mofetil;
^{l} Number of patients on Azathioprine;
^{m} Number of patients on steroids;
ⁿ Number of patients treated by antibody induction therapy.
HC, Healthy Control;
Tol-DF, Tolerant-DrugFree;

TABLE 2
Clinical and natural history summary to Tol-DF patients of the training set (IOT cohort).

| | | | | | | | | | | | Reason for | |
|---|---|---|---|---|---|---|---|---|---|---|---|---|
| | | | | | | | | | | Other | stopping | |
| | | Cause of | Post- | | | Donor | HLA- | Yr IS | Tx | sensitisation | Immuno- | |
| Age ^{a} | Gender | renal failure | Tx ^{b} | eGFR ^{c} | Lymph ^{d} | Type | MM ^{e} | free ^{f} | No ^{g} | events | suppression | Country |
| 40 | male | Glomerulo- | 6 | 79.2 | 1.900 | Cadaveric | 0 | 5 | 2 | Tr, PRA | Stopped due to | UK |
| | | nephritis | | | | | | | | Pre Tx = 80%, | Neck Cancer | |
| | | | | | | | | | | Peak PRA = 92% | | |
| 57 | male | Drug-induced | 4 | 76.4 | N/D | Living- | 0 | 3 | 2 | none recorded | Received HM Tx | UK |
| | | post-leukaemia | | | | related | | | | | from same donor | |
| | | therapy | | | | | | | | | before kidney, | |
| | | | | | | | | | | | had haemopoietic | |
| | | | | | | | | | | | chimerism | |
| 60 | male | Biology | 18 | 79.6 | 2.540 | Cadaveric | 8 | 3 | 1 | Tr, PRA | Self-weaning | France |
| | | Uncertain | | | | | | | | Pre Tx = 0%, | process over | |
| | | | | | | | | | | PeakPRA = 0% | some time | |
| 75 | male | IgA | 33 | 45.7 | 1.273 | Cadaveric | 3 | 21 | 2 | Tr | Self-weaning | France |
| | | Nephropathy | | | | | | | | | process over | |
| | | | | | | | | | | | 4 years | |
| 56 | male | Cystic/ | 9 | 60.8 | 1.700 | Cadaveric | 4 | 3 | 1 | Tr, PRA | Stopped suddenly | Czech |
| | | Polycystic | | | | | | | | Pre Tx = 0, | due to 3 weeks | Republic |
| | | Kidney disease | | | | | | | | Peak PRA = 2, | local floods | |
| | | | | | | | | | | ACR IIA | | |
| 48 | male | urological/ | 29 | 76.2 | 1.440 | Cadaveric | 4 | 11 | 1 | Tr | Self-weaned | UK |
| | | neuropathic | | | | | | | | | and stopped | |
| 54 | male | Glomerulo- | 18 | 56.0 | 1.855 | Living- | 2 | 12 | 1 | none recorded | Self-weaned | Italy |
| | | nephritis | | | | related | | | | | and stopped | |
| 43 | female | Obstructive/ | 5 | 72.7 | 1.300 | Cadaveric | 5 | 3 | 2 | none recorded | Unknown | France |
| | | Reflux | | | | | | | | | | |
| | | Nephropathy | | | | | | | | | | |
| 29 | female | Wegener's | 11 | 84.5 | N/D | Cadaveric | 5 | 2 | 1 | PRA Pre Tx and | Self-weaning | Switzerland |
| | | granulomatosis | | | | | | | | PeakPRA = 0% | process over | |
| | | | | | | | | | | | 1.5 years | |
| 34 | male | Hypertension | 12 | 50.1 | 2.170 | Living- | 1 | 2 | 1 | Tr, | Self-weaning | UK |
| | | | | | | related | | | | PeakPRA = 9% | process over | |
| | | | | | | | | | | | some time | |
| 50 | male | Glomerulo- | 29 | 84.0 | 2.400 | Living- | 6 | 2 | 1 | Tr, | Self-weaning | UK |
| | | nephritis | | | | related | | | | PeakPRA = 0% | process | |

^{a} Age (years);
^{b} Time post-transplantation (years);
^{c} estimated Glomerular Filtration Rate using MDRD function, http://nephron.org/mdrd_gfr_si;
^{d} Peripheral blood lymphocyte counts (×10⁹ cells per L);
^{e} Number of HLA A, B, C, DR, and DQ mismatches between donor and recipient (maximum 10);
^{f} Years patient has been free of all immunosuppressive drugs;
^{g} Transplant number;
Tr: Patient received more than 1 blood transfusion pre-transplant;
PRA, panel reactive antibodies pre Tx:
PRA >1% recorded before transplantation;
Peak PRA: any historic PRA >1% recorded;
ACR IIA: 1 episode of biopsy proven Acute Cellular Rejection of Banff score IIA.
N/D: No data.

TABLE 3
Clinical and demographic characteristics of the test set (ITN cohort)

| | | | % | Post- | | | % 1st | HLA- |
|---|---|---|---|---|---|---|---|---|
| | N | Age ^{a} | Female ^{b} | Tx ^{c} | CRT ^{d} | WBC ^{e} | Tx ^{f} | MM ^{g} |
| HC | 31 | (18-55) | | | | | | |
| Tol-DF | 24 | 51 | 36 | 19 | 88.0 | 6.5 | 73 | 0.0 |
| | | (45-60) | | (13-29) | (79-132) | (3.6-13.8) | | |
| Mono | 11 | 57 | 36 | 12 | 140.8 | 8.0 | 91 | 1.5 |
| | | (48-63) | | (0-38) | (123-158) | (4.8-18.9) | | |
| s-CNI | 34 | 44 | 45 | 6 | 123.2 | 7.8 | 97 | 4.0 |
| | | (39-55) | | (3-17) | (62-246) | (3.7-14.3) | | |
| CAN | 20 | 51 | 37 | 5 | 246.0 | 7.1 | 58 | 4.0 |
| | | (45-57) | | (4-8) | (220-299) | (2.5-20.4) | | |
| All pats | 89 | 50 | 40 | 8 | 132.0 | 7.8 | 81 | 3.0 |
| | | (42-57) | | (0-42) | (106-194) | (2.5-20.4) | | |

^{a, c, d, e,} Medians and interquartile range are shown.
^{a} Age in years;
^{b} Percentage of females;
^{c} Time post-transplantation (years);
^{d} Serum creatinine values (normal range 60-105 μmol/L);
^{e} White blood cell count (×10₉ cells per L);
^{f} Percentage of patients with their first transplant;
^{g} Median number of HLA A, B, and DR, mismatches between donor and recipient (maximum 6).
HC, Healthy Control;
Tol-DF, Tolerant-DrugFree;
Mono, Monotherapy;
s-CNI, Stable-CalcineurinInhibitor;
CAN, Chronic Allograft Nephropathy.

TABLE 4
List of top ranked significant genes within the training set and their annotation enrichment.

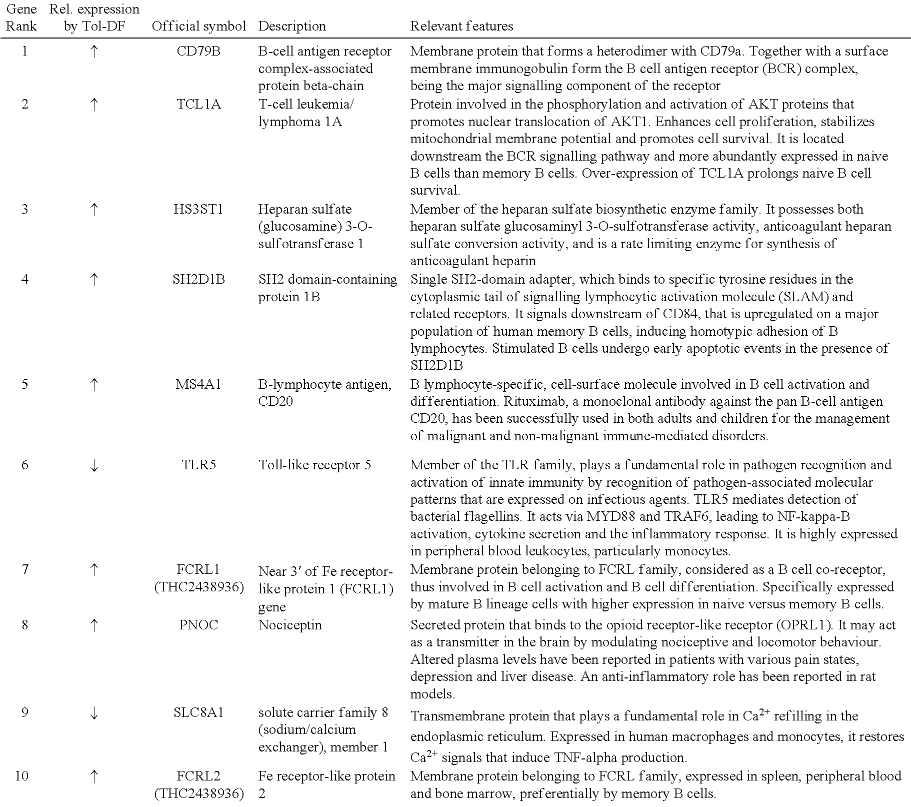

Genes shaded in grey indicate B cell related genes.
Rel. expression by Tol-DF; relative gene expression, upregulation (↑) or downregulation (↓) by Tol-DF group (Median values for fold difference in gene expression for each group available in Table 8).

TABLE 5
Two-sided p values for Mann-Whitney U tests performed between nontolerant patient groups of the training set and
test set when analysing the percentage distribution of peripheral blood lymphocyte subsets by flow cytometry.

| | Training Set | | | | | | | | | |
|---|---|---|---|---|---|---|---|---|---|---|
| | HC vs | HC vs | HC vs | HC vs | s-LP vs | s-LP vs | s-LP vs | s-nCNI vs | s-nCNI vs | s-CNI vs |
| | s-LP | s-nCNI | s-CNI | CR | s-nCNI | s-CNI | CR | s-CNI | CR | CR |
| % T cells | n.s. | 0.044 | n.s. | n.s. | 0.03 | 0.028 | n.s. | n.s. | n.s. | n.s. |
| % B cells | n.s. | 0.001 | n.s. | n.s. | n.s. | n.s. | n.s. | n.s. | n.s. | n.s. |
| % NK cells | n.s. | n.s. | n.s. | n.s. | n.s. | n.s. | n.s. | n.s. | n.s. | n.s. |
| Ratio B/T | n.s. | 0.002 | n.s. | n.s. | 0.044 | n.s. | n.s. | n.s. | n.s. | n.s. |
| % CD4⁺CD25^{int} | n.s. | n.s. | n.s. | n.s. | n.s. | n.s. | n.s. | n.s. | n.s. | n.s. |
| % CD4⁺CD25^{hi} | n.s. | n.s. | n.s. | n.s. | n.s. | 0.026 | n.s. | n.s. | n.s. | n.s. |

Test Set

| | HC vs | HC vs | HC vs | Mono vs | Mono vs | s-CNI vs |
|---|---|---|---|---|---|---|
| | Mono | s-CNI | CAN | s-CNI | CAN | CAN |
| % T cells | n.s. | 0.006 | n.s. | 0.025 | n.s. | 0.033 |
| % B cells | <0.001 | <0.001 | 0.046 | n.s. | n.s. | n.s. |
| % NK cells | n.s. | n.s. | n.s. | 0.009 | n.s. | n.s. |
| Ratio B/T | <0.001 | 0.001 | n.s. | n.s. | n.s. | n.s. |
| % CD4⁺CD25^{int} | 0.016 | 0.002 | 0.005 | n.s. | n.s. | n.s. |
| % CD4⁺CD25^{hi} | 0.001 | 0.004 | n.s. | n.s. | n.s. | n.s. |

TABLE 6
Two-sided p values for the Mann-Whitney U test performed on the calculated ratio of FoxP3
and _1,2-mannosidase expression between non-tolerant patient groups of both study cohorts.

| | Training Set | | | | | | | | | |
|---|---|---|---|---|---|---|---|---|---|---|
| | HC vs | HC vs | HC vs | HC vs | s-LP vs | s-LP vs | s-LP vs | s-nCNI vs | s-nCNI vs | s-CNI vs |
| | s-LP | s-nCNI | s-CNI | CR | s-nCNI | s-CNI | CR | s-CNI | CR | CR |
| P values | 0.004 | <0.001 | <0.001 | <0.001 | n.s. | 0.005 | <0.001 | n.s. | 0.013 | 0.002 |

Test Set

| | HC vs | HC vs | HC vs | Mono vs | Mono vs | s-CNI vs |
|---|---|---|---|---|---|---|
| | Mono | s-CNI | CAN | s-CNI | CAN | CAN |
| P values | 0.004 | <0.001 | <0.001 | n.s. | 0.052 | n.s. |

TABLE 7
List of 174 probes common to both training and test sets
identified to have significant differential expression by study
groups following four-class analysis. Ranked on p values by
Kruskal-Wallis test with adjustment for False Discovery Rate
(FDR) at 1%.
Common gene list

| Training | | | Test set |
|---|---|---|---|
| set Rank | Gene Name | Probe ID | Rank |
| 1 | CD79B | A_23_P207201_riset1 | 12 |
| 2 | TCL1A | A_23_P357717_riset1 | 1 |
| 3 | HS3ST1 | A_23_P121657_riset1 | 3 |
| 4 | TCL1A | MIL_PPPID394307780_riset1 | 2 |
| 5 | SH2D1B | A_23_P351148_riset1 | 352 |
| 6 | MS4A1 | MIL_PPPID394453510_riset1 | 57 |
| 7 | TLR5 | A_23_P85903_riset1 | 97 |
| 8 | THC2438936 | A_32_P71876_riset1 | 52 |
| 9 | PNOC | A_23_P253321_riset1 | 53 |
| 10 | SLC8A1 | A_32_P147078_riset1 | 736 |
| 11 | FCRL2 | A_23_P160751_riset1 | 43 |
| 12 | BLK | BLK_riset2 | 111 |
| 13 | PCDH9 | A_24_P187218_riset1 | 9 |
| 14 | CD200 | A_23_P121480_riset1 | 4 |
| 15 | THC2317432 | A_32_P124728_riset1 | 10 |
| 16 | BCL11A | A_24_P402588_riset1 | 251 |
| 17 | TSPAN3 | A_23_P14804_riset1 | 80 |
| 18 | CD79A | CD79A_riset2_piqor | 29 |
| 19 | CASP5 | CASP5_riset2 | 493 |
| 20 | FCRL5 | A_23_P201211_riset1 | 301 |
| 21 | ILIRL2 | A_23_P56604_riset1 | 930 |
| 22 | EBI2 | A_23_P25566_riset1 | 82 |
| 23 | BLNK | A_24_P64344_riset1 | 20 |
| 25 | RNF24 | A_23_P120533_riset1 | 784 |
| 26 | DIMT1L | A_23_P58529_riset1 | 494 |
| 27 | EPO | A_23_P145664_riset1 | 807 |
| 28 | ZAK | A_23_P318300_riset1 | 244 |
| 29 | CD40 | A_23_P57036_riset1 | 205 |
| 31 | IGHM | A_24_P417352_riset1 | 13 |
| 33 | SH2D1B | A_32_P324533_riset1 | 329 |
| 34 | AKR1C3 | A_23_P138543_riset1 | 106 |
| 35 | CASD1 | A_24_P69379_riset1 | 163 |
| 36 | FCRLA | A_23_P46037_riset1 | 56 |
| 37 | BTLA | MIL_PPPID399200354_riset1 | 28 |
| 39 | CLCN5 | CLCN5_riset2 | 959 |
| 40 | LHFPL2 | A_23_P255104_riset1 | 65 |
| 41 | ARG1 | A_23_P111321_riset1 | 360 |
| 42 | ANXA3 | A_23_P121716_riset1 | 231 |
| 43 | HLA-DOA | A_32_P356316_riset1 | 74 |
| 44 | KLRF1 | A_32_P158966_riset1 | 815 |
| 45 | CD22 | CD22_riset2 | 121 |
| 47 | PLXDC2 | A_32_P187518_rev_riset1 | 60 |
| 48 | BCL7A | A_24_P203056_riset1 | 59 |
| 50 | ADAMTS4 | A_23_P360754_riset1 | 5 |
| 54 | HLA-DOB | A_23_P30736_riset1 | 128 |
| 55 | CD163 | A_23_P33723_riset1 | 637 |
| 56 | FLJ32866 | MIL_PPPID400032869_riset1 | 437 |
| 57 | EBF1 | A_24_P156501_riset1 | 102 |
| 58 | GPR92 | A_23_P204375_riset1 | 1182 |
| 60 | PEX3 | A_23_P259328_riset1 | 413 |
| 62 | CD83 | A_23_P70670_riset1 | 123 |
| 63 | GPR120 | A_24_P129588_riset1 | 833 |
| 66 | ASPH | A_24_P295245_riset1 | 214 |
| 67 | MCTP1 | A_24_P212481_riset1 | 19 |
| 68 | SORT1 | A_23_P201587_riset1 | 83 |
| 70 | TMEM49 | A_32_P9753_riset1 | 58 |
| 71 | MBOAT2 | A_23_P370635_riset1 | 110 |
| 75 | SIRPA | A_24_P267619_riset1 | 778 |
| 76 | PDHX | A_23_P36266_riset1 | 196 |
| 77 | STAT3 | A_23_P100790_riset1 | 1250 |
| 78 | SLP1 | A_23_P91230_riset1 | 389 |
| 81 | RET | MIL_PPPID397416187_riset1 | 1074 |
| 82 | GPR84 | A_23_P25155_riset1 | 125 |
| 83 | ITK | ITK_riset2 | 99 |
| 84 | AIM2 | A_32_P44394_riset1 | 218 |
| 87 | TNFAIP8 | TNFAIP8_riset2 | 132 |
| 91 | KCNJ2 | A_23_P329261_riset1 | 112 |
| 92 | BNIP3 | A_23_P138635_riset1 | 85 |
| 93 | GALNT14 | A_23_P67847_riset1 | 333 |
| 94 | TGDS | A_23_P65217_riset1 | 78 |
| 96 | DNAJC19 | A_23_P121396_riset1 | 638 |
| 100 | THC2380706 | A_24_P214556_riset1 | 714 |
| 101 | C1ORF19 | A_23_P104022_riset1 | 891 |
| 103 | LITAF | A_23_P3532_riset1 | 1226 |
| 106 | SLC25A37 | A_23_P393115_riset1 | 504 |
| 108 | EDG1 | A_23_P160117_riset1 | 109 |
| 110 | TNFAIP6 | A_23_P165624_riset1 | 496 |
| 112 | TMTC3 | A_24_P944222_riset1 | 331 |
| 113 | MNAT1 | A_23_P32615_riset1 | 541 |
| 114 | ZNF181 | A_23_P50735_riset1 | 770 |
| 115 | MYC | MIL_PPPID404158649_riset2 | 1072 |
| 116 | CLEC5A | A_23_P71165_riset1 | 607 |
| 117 | GPX7 | A_23_P73972_riset1 | 274 |
| 119 | PPIL3 | A_23_P28213_riset1 | 131 |
| 120 | THUMPD1 | A_24_P90878_riset1 | 317 |
| 121 | MAD2L1 | A_23_P92441_riset1 | 300 |
| 122 | SPTLC2 | A_24_P150486_riset1 | 66 |
| 123 | THAP2 | A_24_P350437_riset1 | 310 |
| 125 | C8ORF60 | A_24_P59387_riset1 | 409 |
| 126 | MXD1 | A_23_P408094_riset1 | 732 |
| 130 | SEPT9 | A_23_P106973_riset1 | 248 |
| 131 | PDCD6 | A_23_P218997_riset1 | 433 |
| 133 | IL10RA | A_24_P107303_riset1 | 266 |
| 134 | MAGEH1 | A_23_P34144_riset1 | 98 |
| 136 | SLC25A37 | A_32_P197026_riset1 | 119 |
| 137 | CARD6 | A_23_P41854_riset1 | 926 |
| 138 | ZFP2 | A_23_P133359_riset1 | 1075 |
| 139 | PTMA | A_32_P746413_riset1 | 392 |
| 140 | BACE2 | A_23_P154875_riset1 | 735 |
| 141 | CHST2 | CHST2_riset2 | 359 |
| 143 | BCKDHB | A_24_P239664_riset1 | 335 |
| 144 | MTX2 | A_23_P120335_riset1 | 716 |
| 148 | CD7 | A_23_P118862_riset1 | 804 |
| 149 | ACOT2 | A_23_P3111_riset1 | 577 |
| 153 | CD40LG | A_23_P62220_riset1 | 275 |
| 155 | FANK1 | A_23_P115785_riset1 | 48 |
| 156 | DNMT1 | A_23_P27490_riset1 | 240 |
| 157 | AK3L1 | MIL_PPPID394453462_riset1 | 1122 |
| 158 | RFC4 | A_23_P18196_riset1 | 87 |
| 159 | BTG1 | A_23_P87560_riset1 | 17 |
| 160 | SPON1 | A_32_P133072_riset1 | 103 |
| 162 | ATL3 | A_23_P105028_riset1 | 51 |
| 165 | AFF3 | A_23_P373464_riset1 | 690 |
| 166 | MCEE | A_23_P56654_riset1 | 1013 |
| 167 | NLRC4 | A_23_P119835_riset1 | 779 |
| 169 | DTX1 | A_24_P290751_riset1 | 115 |
| 170 | TRIM4 | A_23_P500601_riset1 | 227 |
| 171 | TMEM106B | A_32_P353072_riset1 | 293 |
| 176 | FCAR | A_24_P348265_riset1 | 241 |
| 178 | VCPIP1 | A_32_P74366_riset1 | 509 |
| 179 | SATB1 | SATB1_riset2 | 338 |
| 181 | PDLIM7 | A_24_P41882_riset1 | 162 |
| 184 | IL23A | A_23_P76078_riset1 | 152 |
| 187 | XBP1 | A_23_P120845_riset1 | 443 |
| 188 | SOCS3 | A_23_P207058_riset1 | 831 |
| 189 | SFRS10 | A_24_P145911_riset1 | 209 |
| 190 | TNFSF10 | A_23_P121253_riset1 | 1029 |
| 195 | RNASE2 | A_23_P151638_riset1 | 178 |
| 196 | MAP2K6 | A_23_P207445_riset1 | 1048 |
| 198 | LYRM7 | A_32_P18159_riset1 | 1375 |
| 199 | AK124263 | A_24_P538708_riset1 | 911 |
| 200 | MYT1:PCMTD2 | A_23_P210829_riset1 | 995 |
| 201 | CD96 | A_23_P44154_riset1 | 533 |
| 203 | PCGF6 | A_23_P115703_riset1 | 454 |
| 204 | IGKV3-20 | A_23_P21800_riset1 | 11 |
| 205 | CARD11 | A_23_P82324_riset1 | 502 |
| 206 | IFT80 | A_23_P316150_riset1 | 375 |
| 207 | SLAMF1 | SLAMF1_riset2 | 62 |
| 208 | SIP1 | A_23_P88194_riset1 | 516 |
| 209 | FAIM | A_23_P253932_riset1 | 590 |
| 210 | NEK1 | A_23_P124427_riset1 | 1368 |
| 211 | IL1R2 | A_23_P79398_riset1 | 545 |
| 212 | HOXB2 | A_23_P107283_riset1 | 142 |
| 214 | FLT3 | MIL_PPPID394308044_riset1 | 345 |
| 216 | SLC26A8 | A_23_P30944_riset1 | 264 |
| 219 | FCGR1A | A_23_P63395_riset1 | 318 |
| 220 | LGALS8 | LGALS8_riset2 | 1102 |
| 222 | CLIC3 | A_23_P254654_riset1 | 1317 |
| 223 | BEX4 | A_23_P114391_riset1 | 176 |
| 224 | SLC11A1 | A_32_P436132_riset1 | 1086 |
| 225 | BMP2K | A_24_P86240_riset1 | 27 |
| 226 | SFT2D3 | A_23_P5566_riset1 | 370 |
| 227 | C5ORF28 | A_23_P121875_riset1 | 400 |
| 230 | PHLDB2 | A_24_P240166_riset1 | 1092 |
| 231 | A_23_P399604 | A_23_P399604_riset1 | 401 |
| 232 | GTL3 | A_23_P3514_riset1 | 616 |
| 233 | ITSN2 | MIL_PPPID399200362_riset1 | 733 |
| 234 | ARHGEF12 | A_24_P175783_riset1 | 546 |
| 236 | MUC8 | MUC8_riset2_piqor | 1198 |
| 238 | FLJ22662 | A_23_P87709_riset1 | 267 |
| 239 | APEX1 | A_23_P151649_riset1 | 585 |
| 240 | RPS4Y1 | A_23_P259314_riset1 | 1132 |
| 241 | LCK | LCK_riset2 | 207 |
| 242 | LOC642897 | A_24_P84401_riset1 | 933 |
| 244 | SLC26A8 | A_23_P30950_riset1 | 161 |
| 246 | CD247 | A_23_P34676_riset1 | 16 |
| 247 | UPF3A | A_32_P202778_riset1 | 282 |
| 248 | RGS1 | A_23_P97141_riset1 | 22 |
| 249 | RBM3 | A_23_P148308_riset1_piqor | 619 |
| 250 | POLR3H | A_24_P322847_riset1 | 744 |
| 251 | RHOH | A_23_P58132_riset1 | 49 |
| 254 | LETMD1 | A_23_P117037_riset1 | 137 |
| 257 | LDHB | A_23_P53476_riset1 | 77 |
| 259 | TRA@ | A_23_P258504_riset1 | 312 |

TABLE 8
Median of log-fold change in expression (relative to median of all samples) of differentially
expressed probes detected in patient groups of the training set, ranked on 1% FDR.
Plots showing the fold expression for each patient group of the training and test
sets, for all genes detected to be significantly differentially expressed available
in online material. Stable; combined groups s-CNI, s-nCNI, s-LP.

| Rank | Probe | Probe ID | HC | Tol-DF | Stable | CR |
|---|---|---|---|---|---|---|
| 1 | CD79B | A_23_P207201_riset1 | 1.024 | 0.618 | −0.2375 | −0.319 |
| 2 | TCL1A | A_23_P357717_riset1 | 1.6525 | 1.062 | −0.41 | 0.0965 |
| 3 | HS3ST1 | A_23_P121657_riset1 | 1.161 | 0.995 | −0.8895 | −1.8945 |
| 4 | TCL1A | MIL_PPPID394307780_riset1 | 1.645 | 0.994 | −0.38 | 0.121 |
| 5 | SH2D1B | A_23_P351148_riset1 | 1.085 | 0.76 | −0.198 | −0.0445 |
| 6 | MS4A1 | MIL_PPPID394453510_riset1 | 0.9025 | 0.763 | −0.2725 | −0.108 |
| 7 | TLR5 | A_23_P85903_riset1 | −0.7895 | −0.341 | 0.1265 | 0.36 |
| 8 | THC24389 | A_32_P71876_riset1 | 1.1145 | 0.517 | −1.2275 | −0.9745 |
| 9 | PNOC | A_23_P253321_riset1 | 0.633 | 0.585 | −0.2725 | 0.0465 |
| 10 | SLC8A1 | A_32_P147078_riset1 | −0.591 | −0.451 | 0.04 | 0.4665 |
| 11 | FCRL2 | A_23_P160751_riset1 | 1.067 | 0.554 | −0.845 | −0.9725 |

TABLE 9
Annotation enrichment of genes with significant differential expression and common to the
training and test sets. Listed genes are those identified to be significantly associated with the
listed annotated pathways. Shaded columns indicate B cell related pathways.

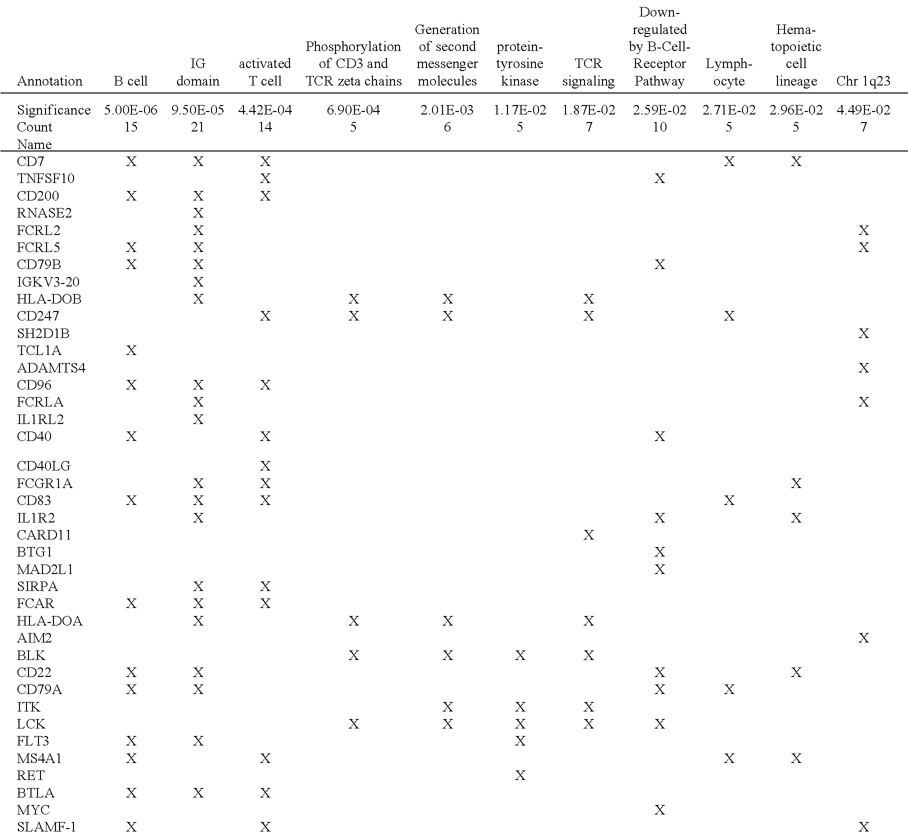

### REFERENCES

1. Christensen, L. L. et al., Tissue Antigens, 51:637-644, 1998.
2. Owens, M. L. et al., Arch Surg., 110:1450-1451, 1975.
3. Roussey-Kesler, G. et al., AM J Transplant, 6:736-746, 2006.
4. Uehling, D. T. et al., Surgery, 79:278-282, 1976.
5. Strober. S. et al., Transplantation, 69:1549-1554, 2000.
6. Takatsuki, M. et al., Transplantation, 72:449-454, 2001.
7. Lerut, J. et al., AM J Transplant, 6:1774-1780, 2006.
8. Padiyar, A. et al., Prim Care, 35:433-450, v, 2008.
9. Miyara, M. et al., Immunity, 2009 30:899-911.
10. Shevach, E. M. et al., J Exp Med, 193:F41-46, 2001.
11. Sawitzki, B. et al., AM J Transplant, 7:1091-1102, 2007.
12. Striminer, K. et al., BMC Bioinformatics, 9:303, 2008.
13. Louis, S. et al., Transplantation, 81:398-407, 2006.
14. Afzali, B. et al., Clin Exp Immunol, 148:32-46, 2007.
15. Ely, L. K. et al., Curr Opin Immunol, 20:575-580, 2008.
16. Lechler, R. I. et al., Nat Rev Immunol, 3:147-158, 2003.
17. Deng, S. et al., J Immunol, 178:6028-6032, 2007.
18. Reichardt, P. et al., Blood, 110:1519-1529, 2007.
19. Porcheray, F. et al., AM J Transplant, 9:2126-2135, 2009.
20. de Haan, A. et al., Transplantation, 69:1434-1439, 2000.
21. Hornick, P. I. et al., Circulation, 97:1257-1263, 1998.
22. Beik, A. I. et al., Nephrol Dial Transplant, 12:1949-1955, 1997.
23. Martinez-Llordella, M. et al., J Clin Invest, 118:2845-2857, 2008.
24. Martinez-Llordella, M. et al., AM J Transplant, 7:309-319, 2007.
25. Braid, C. et al., J Cell Biochem, 2007.
26. Brouard, S. et al., Proc Natl Acad Sci USA, 104:15448-15453, 2007.
27. Asare, A. L. et al., BMC Genomics, 9:474, 2008.
28. Gunther, O. P. et al., Transplantation, 88:942-951, 2009.
29. Sawitzki, B. et al., Curr Opin Organ Transplant, 14:650-655, 2009.
30. Carsetti, R. et al., Immunol Rev, 197:179-191, 2004.
31. Garin, M. I. et al., Blood, 109:2058-2065, 2007.
32. Ozawa, M. et al., Tissue Antigens, 69 Suppl 1:174-179, 2007.
33. Game, D. S. et al., J Am Soc Nephrol, 14:1652-1661, 2003.
34. Weng, L. et al., Bioinformatics, 22:1111-1121, 2006.
35. Edgar, R. et al., Nucleic Acids Res, 30:207-210, 2002.
36. Mazariegos G V. et al., Transplantation, 27; 73(8):1342-5, 2002.
37. Mazariegos G V. et al., Am J Transplantation, 3(6):689-96, June 2003
38. Martinez-Llordella M. et al., Am J Transplant, 7(2):309-19, February 2007.
39. Martinez-Llordella M. et al., J Clin Invest, 118(8):2845-57, August 2008.
40. Brouard S. et al., Am J Transplant, 5(2):330-40, February 2005.
41. Brouard S. et al., Proc Natl Acad Sci USA, 25; 104(39):15448-53, September 2007.
42. Louis S. et al., Transplantation, 15; 81(3):398-407, February 2006.
43. Velásquez S Y. et al., Transpl Int., 23(1):54-63, January 2010.
44. Louvet C. et al., Am J Transplant, 5(9):2143-53, September 2005.
45. Baeten et al., J. Am. Soc. Nephrol., 17: 294.104, 2006

1. A method of determining an individual's immunological tolerance to a kidney organ transplantation comprising determining the level of expression of at least 2 genes selected from the group consisting of TLR5, PNOC, SH2D1B, CD79B, TCL1A, HS3ST1, MS4A1, FCRL1, SLC8A1 and FCRL2 in a sample obtained from the individual with the transplanted kidney.

2. The method of claim 1, wherein an individual is determined to have immunological tolerance to the kidney organ transplantation when the level of expression of SH2D1B, PNOC, CD79B, TCL1A, HS3ST1, MS4A1, FCRL1 and FCRL2 is higher than a normal level, and wherein an individual is determined to have immunological tolerance when the level of expression of TLR5 and SLC8A1 is lower than a normal level.

3. The method of claim 1, wherein the level of expression of genes TLRS, PNOC and SH2D1B in a sample obtained from the individual is determined.

4. The method of claim 3, wherein a positive prediction of an individual's tolerance to an organ transplantation is given when a high level of expression of SH2D1 B and PNOC and a low level of expression on TLR5S is determined.

5. The method claim 3, wherein the expression level of one or more of the following genes CD79B, TCL1A, HS3ST1, MS4A1, FCRL1, SLC8A1 and FCRL2 is additionally determined.

6. The method of claim 1, wherein the method further comprises detecting the level of B cells and NK cells, wherein a raised level of such cells is indicative of immunological tolerance.

7. The method of claim 1 wherein the method further comprises determining the level of CD4+CD25^{int} T cells, wherein a reduced level of such cells relative to total CD4+ T cells is indicative of immunological tolerance.

8. The method of claim 1, wherein the method further comprises determining the level of donor specific CD4+ T cells, wherein a reduced level of such cells is indicative of immunological tolerance.

9. The method of claim 1, wherein the method further comprises determining the ratio of expression levels of FoxP3 to α-1,2-mannosidase gene of CD4+ T cells, wherein a high ratio is indicative of immunological tolerance.

10. The method of claim 1, wherein the method further comprises determining the ratio of CD19+ to CD3+ cells, wherein a high ratio is indicative of immunological tolerance.

11. The method of claim 1, wherein the expression levels of beta-actin and/or HRPT are used as controls.

12. A sensor for detecting expression levels, comprising one or more nucleic acid probes specific for at least 2 genes selected from the group consisting of TLR5, PNOC, SH2D1B, CD79B, TCL1A, HS3ST1, MS4A1, FCRL1, SLC8A1, and FCRL2.

13. A sensor for determining expression levels, comprising one or more nucleic acid probes specific for each of the TLRS, PNOC and SH2D1B genes.

14. The sensor of claim 13, which is for detecting the expression of one or more of the following genes CD79H, TCL1A, HS3ST1, MS4A 1, FCRL1, SLC8A1 and FCRL2.

15. A kit comprising reagents for detecting the level of expression, comprising one or more nucleic acid probes or primers specific for at least 2 genes selected from the group consisting of TLR5, PNOC, SH2D1B, CD79B, TCL1A, HS3ST1, MS4A1, FCRL1, SLC8A1 and FCRL2.

16. A kit comprising reagents for detecting the level of expression, comprising one or more nucleic acid probes or primers specific for the TLR5, PNOC and SH2D1B genes.

17. The kit of claim 16 that further comprises reagents for detecting the level of expression of one or more of the following genes CD79B, TCL1A, HS3ST1, MS4A 1, FCRL1, SLC8A 1 and FCRL2.

18. The kit of claim 15, which comprises reagents for detecting the level of gene expression of the genes by RT-PCR.

19. The kit of claim 16, which comprises reagents for detecting the level of gene expression of the genes by RT-PCR.

20. The kit of claim 17, which comprises reagents for detecting the level of gene expression of the genes by RT-PCR.
